(19) United States
(12) Patent Application Publication
(10) Pub. No.: US 2025/0302298 A1
(43) Pub. Date: Oct. 2, 2025

(54) OPHTHALMIC MEASUREMENT DEVICE

(71) Applicant: NIDEK CO., LTD., Aichi (JP)

(72) Inventors: Michihiro Takii, Gamagori (JP); Mikio Kurachi, Gamagori (JP); Naoki Isogai, Gamagori (JP); Hiroshi Ohta, Gamagori (JP)

(73) Assignee: NIDEK CO., LTD., Aichi (JP)

(21) Appl. No.: 19/239,995

(22) Filed: Jun. 17, 2025

Related U.S. Application Data
(63) Continuation of application No. PCT/JP2023/044315, filed on Dec. 11, 2023.

(30) Foreign Application Priority Data
Jan. 17, 2023 (JP) 2023-005190
Jan. 17, 2023 (JP) 2023-005191
Jan. 17, 2023 (JP) 2023-005192
Jul. 28, 2023 (JP) 2023-123150

(51) Int. Cl. A61B 3/028; A61B 3/00

(52) U.S. Cl. CPC A61B 3/028; A61B 3/0075; A61B 3/0091

(57) ABSTRACT

An ophthalmic measurement device is provided and includes: a measurement optical system, projecting measurement light onto a fundus of an eye to be examined and receive a reflected light from the fundus by a light receiving element; a conversion member, arranged at a pupil conjugate position of the eye to be examined in the measurement optical system, converting the reflected light into multiple ring images; a limiting part, including a limiting member arranged at a conjugate position of the conversion member or in the vicinity of the conversion member, and changing a measurement region on the eye to be examined by limiting some of the ring images with the limiting member; a limiting controller, controlling the limiting part and causing the measurement region to change in at least two patterns; and a processor, processing the ring images and obtaining eye refractive power information.

### CROSS-REFERENCE TO RELATED APPLICATION

The present application is a continuation of PCT/JP2023/044315, filed on Dec. 11, 2023, and is related to and claims priorities from Japanese Patent Application No. 2023-005190 filed on Jan. 17, 2023, Japanese Patent Application No. 2023-005191 filed on Jan. 17, 2023, Japanese Patent Application No. 2023-005192 filed on Jan. 17, 2023, and Japanese Patent Application No. 2023-123150 filed on Jul. 28, 2023. The entire contents of the aforementioned applications are hereby incorporated by reference herein.

### TECHNICAL FIELD

The disclosure relates to an ophthalmic measurement device that measures an eye refractive power distribution of an eye to be examined.

### BACKGROUND

As ophthalmic measurement devices that measure eye refractive power distribution of an eye to be examined, there have been proposed: a system that projects a slit light beam onto the fundus of the eye to be examined and utilizes a phase difference signal when a reflected light beam is detected by a light receiving element (for example, see Patent Document 1), a system that converts a reflected light beam from the fundus of the eye to be examined into measurement index images composed of a large number of point images (including the use of wavefront sensors and microlens arrays) and receives the measurement index images by a light receiving element (for example, see Patent Document 2), and a system that converts a reflected light beam from the fundus of the eye to be examined into measurement index images composed of multiple ring images and receives the measurement index images by a light receiving element (for example, see Patent Document 3). For instance, the result of measuring the eye refractive power distribution of an eye to be examined is used for corneal correction surgery in which the corneal shape is changed by laser light.

### RELATED ART DOCUMENTS

### Patent Documents

[Patent Document 1] Japanese Patent Application Laid-Open No. H10-108837
[Patent Document 2] Japanese Patent Application Laid-Open No. 2008-113810
[Patent Document 3] Japanese Patent Application Laid-Open No. 2005-103103

However, to obtain more accurate and more appropriate measurement results, further improvements are desired. In the system utilizing multiple ring images, for example, depending on the condition of the eye to be examined, or due to dense ring images received by the light receiving element, it may be difficult to distinguish adjacent ring images. In this case, it becomes difficult to obtain appropriate measurement results.

Besides, in the system utilizing multiple ring images, for example, it is necessary to identify the number of each ring image received by the light receiving element among corresponding measurement regions on the eye to be examined. However, depending on the condition of the eye to be examined, the ring images on the light receiving element may be disturbed or the signal level may be low, making it difficult to distinguish the number that each ring image corresponds to. If the ring image is incorrectly distinguished, it becomes difficult to obtain appropriate measurement results.

In addition, in the system utilizing ring images, for example, the pupil edge (iris edge) may overlap with the width of the corresponding ring-shaped measurement region on the pupil, causing vignetting of the measurement light beam passing through the pupil. In this case, if the vignetting of the measurement light beam is not taken into consideration, correct eye refractive power information (including cases of aberration information) at the measurement position (measurement region) on the pupil cannot be obtained.

The disclosure provides an ophthalmic measurement device that is capable of obtaining more accurate and more appropriate measurement results.

### SUMMARY

An ophthalmic measurement device according to an exemplary embodiment of the disclosure is an ophthalmic measurement device that measures eye refractive power distribution of an eye to be examined. The ophthalmic measurement device includes: a measurement optical system including a light projecting optical system that projects a measurement light onto a fundus of the eye to be examined, and a light receiving optical system that receives a reflected light of the measurement light from the fundus of the eye to be examined by a light receiving element; a conversion member arranged at a pupil conjugate position of the eye to be examined in the measurement optical system, and converting the reflected light from the fundus into a plurality of ring images and causing the ring images to be received by the light receiving element; a limiting part including a limiting member arranged at a conjugate position of the conversion member or in a vicinity of the conversion member, and changing a measurement region on the eye to be examined by limiting, with the limiting member, some of the ring images received by the light receiving element; a limiting controller configured to control the limiting part and cause the measurement region on the eye to be examined that is changed by the limiting member to change in at least two patterns; and a processor configured to process the ring images received by the light receiving element and obtain eye refractive power information.

### BRIEF DESCRIPTION OF THE DRAWINGS

FIG. 1 is a diagram of an external appearance of an ophthalmic measurement device.

FIG. 2 is a schematic diagram of an optical system of an ophthalmic measurement device in the first embodiment.

FIG. 3 is a diagram showing an example of a ring lens of the disclosure.

FIG. 4A is an example of a mask member used in a preliminary measurement stage.

FIG. 4B is an example of a mask member used in a preliminary measurement stage.

FIG. 5A is a diagram explaining the distinction between adjacent ring images.

FIG. 5B is a diagram explaining the distinction between adjacent ring images.

FIG. 5C is a diagram explaining the distinction between adjacent ring images.

FIG. 6A is an example of a mask member that limits to prevent adjacent ring images from being received.

FIG. 6B is an example of a mask member that limits to prevent adjacent ring images from being received.

FIG. 7A is a diagram explaining a measurement of a precise eye refractive power distribution.

FIG. 7B is a diagram explaining a measurement of a precise eye refractive power distribution.

FIG. 7C is a diagram explaining a measurement of a precise eye refractive power distribution.

FIG. 7D is a diagram explaining a measurement of a precise eye refractive power distribution.

FIG. 7E is a diagram explaining a measurement of a precise eye refractive power distribution.

FIG. 8A is a diagram showing an example of a mask member applied to a precision measurement mode of an eye refractive power distribution.

FIG. 8B is a diagram showing an example of a mask member applied to a precision measurement mode of an eye refractive power distribution.

FIG. 9 is a diagram showing an optical system of an ophthalmic measurement device in the second embodiment.

FIG. 10A is an example showing a case where it is difficult to determine the number that a ring image on an imaging element corresponds to, and an example of ring images in a case where internal reflections within the eye or corneal reflections overlap.

FIG. 10B is an example showing a case where it is difficult to determine the number that a ring image on an imaging element corresponds to, and an example of ring images in a case where a spherical power of the eye to be examined is positive.

FIG. 10C is an example showing a case where it is difficult to determine the number that a ring image on the imaging element corresponds to, and an example of ring images in a case where a spherical power of the eye to be examined is negative.

FIG. 11 is a diagram showing an example of a mask member of a mark formation mask.

FIG. 12 is an example of ring images received by an imaging element in a case where a mask member is placed in an optical path of a light receiving optical system.

FIG. 13 is a diagram showing an example in which, compared to FIG. 12, marks are formed to make a first ring image and a second ring image from the inside distinguishable.

FIG. 14A is a diagram explaining an example where a microlens array is used instead of the ring lens.

FIG. 14B is a diagram explaining an example where a microlens array is used instead of the ring lens.

FIG. 14C is a diagram explaining an example where a microlens array is used instead of the ring lens.

FIG. 15A is a diagram explaining a measurement considering a vignetting state of a measurement light beam due to a pupil edge.

FIG. 15B is a diagram explaining a measurement considering a vignetting state of a measurement light beam due to a pupil edge.

FIG. 16A is a diagram explaining a comparison between a pupil region in an anterior segment image and a corresponding measurement region on a pupil.

FIG. 16B is a diagram explaining a comparison between a pupil region in an anterior segment image and a corresponding measurement region on a pupil.

FIG. 17 is a diagram explaining a case of obtaining an eye refractive power information for each meridian direction for each measurement region corresponding to the ring image considering vignetting of the measurement light beam.

FIG. 18 is a diagram explaining a case where an imaging element is disposed at a position away from and behind the focusing point of the ring image.

FIG. 19A is a diagram showing the ring image detected on the acquired image in a case where no vignetting occurs in the measurement light beam in FIG. 18.

FIG. 19B is a diagram showing the ring image detected on the acquired image in a case where vignetting occurs in the measurement light beam in FIG. 18.

FIG. 20A is a diagram explaining a variation example of a mask member that prevents adjacent ring images from being received by the imaging element.

FIG. 20B is a diagram explaining a variation example of a mask member that prevents adjacent ring images from being received by the imaging element.

FIG. 20C is a diagram explaining a variation example of a mask member that prevents adjacent ring images from being received by the imaging element.

FIG. 21 is a diagram showing an example of the first image acquired in a case where the mask member is in the arrangement state of FIG. 20A.

### DESCRIPTION OF THE EMBODIMENTS

### Overview

The following will describe one of the exemplary embodiments with reference to the drawings. The items classified by < > below may be used independently or in relation to each other.

For example, an ophthalmic measurement device (for example, ophthalmic measurement device 10) measures an eye refractive power distribution (including cases of wavefront aberration) of an eye to be examined. For example, the ophthalmic measurement device includes a measurement optical system (for example, measurement optical system 100), a conversion member (for example, ring lens 125, microlens array 125S), and a first processor (for example, control part 50). For example, the ophthalmic measurement device may include a limiting part (for example, mask 130) and a limiting controller (for example, control part 50). For example, the ophthalmic measurement device may include at least one of a diopter corrector (for example, driver 160, drive part 161), a measurement controller (for example, control part 50), and a mode switching part (for example, operation part 17, control part 50). For example, the ophthalmic measurement device may include an alignment detection part (for example, alignment index projection optical system 600, observation optical system 200, control part 50).

For example, the ophthalmic measurement device may include at least one of a mark formation part (for example, mark formation mask 140), a second processor (for example, control part 50), an insertion/removal part (for example, mask drive part 140A), and an insertion/removal controller (for example, control part 50). For example, the ophthalmic measurement device may include at least one of an anterior segment image acquisition part (for example, observation optical system 200) and a third processor (for example, control part 50).

### <Measurement Optical System>

For example, the measurement optical system includes a light projecting optical system (for example, light projecting optical system 110) that projects a measurement light onto a fundus of an eye to be examined, and a light receiving optical system (for example, light receiving optical system 120) that receives a reflected light of the measurement light from the fundus of the eye to be examined by a light receiving element (for example, imaging element 126).

### <Alignment Detection Part>

For example, the alignment detection part detects an alignment state of the measurement optical system with respect to the eye to be examined. For example, the alignment detection part may include an index projection optical system (alignment index projection optical system 600) that projects an alignment index onto the eye to be examined, and a detection optical system (observation optical system 200) that detects the alignment index projected onto the eye to be examined. For example, the alignment detection part detects displacement of alignment deviation in an X direction (left-right direction with respect to the eye to be examined) and a Y direction (up-down direction with respect to the eye to be examined) of the measurement optical system with respect to the eye to be examined based on the detection result of the alignment index projected onto the eye to be examined.

### <Conversion Member>

For example, the conversion member is arranged at a pupil conjugate position of the eye to be examined in the measurement optical system. For example, the conversion member may convert the reflected light of the measurement light from the fundus into measurement index images composed of multiple (for example, three or more) ring images, and cause the measurement index images to be received by the light receiving element. Alternatively, the conversion member may convert the reflected light of the measurement light from the fundus into measurement index images composed of a large number of point images (for example, a large number of point images in three or more circles centered on a measurement optical axis), and cause the measurement index images to be received by the light receiving element. For example, a lens member is used as the conversion member. For example, in a case of converting into multiple ring images, the conversion member may be multiple ring lenses (for example, ring lens 125). For example, the ring lens includes three or more ring-shaped columnar lens portions. For example, in a case of converting into measurement index images composed of a large number of point images, the conversion member may be a microlens array or a Hartmann plate.

### <Limiting Part>

For example, the limiting part has a limiting member (for example, mask member 131, 132, 135, 136, 137, 138, 143, 144, 145) arranged at a conjugate position of the conversion member or in the vicinity of the conversion member. For example, the limiting part may be a mask part. For example, the limiting part changes the measurement region on the eye to be examined by limiting, with the limiting member, some of the measurement index images (for example, ring images) received on the light receiving element by the conversion member. For example, the limiting part may include multiple limiting members (for example, mask members). For example, the limiting part has a drive part (for example, mask drive part 130A), and the multiple limiting members are selectively inserted into and removed from the optical path by the drive part. For example, in a case where the conversion member converts the reflected light from the fundus into measurement index images composed of multiple ring images, and causes the measurement index images to be received by the light receiving element, the limiting part may have at least one of: a first limiting member (for example, mask member 131, 132) that limits the ring images received by the light receiving element to one or two; a second limiting member (for example, mask member 135, 136, 143, 144, 145) that limits so that adjacent ring images in a meridian direction are not received by the light receiving element; and a third limiting member (for example, mask member 137, 138) that divides the ring width of the ring images received by the light receiving element.

In the disclosure, the conjugate position also includes an approximate conjugate position. “Approximate conjugate” means that the position does not need to be perfectly conjugate, and may be conjugate with the accuracy required in relation to measurement precision. In addition, “vicinity” means that the position may be near with the accuracy required in relation to measurement precision.

For example, for the first limiting member, it is possible to use a limiting member with at least two patterns that limits the ring images received by the light receiving element to different diameters. In other words, the first limiting member may be a limiting member with at least two patterns that limits the multiple ring images received on the light receiving element to ring images with at least two different diameters.

For example, for the second limiting member, it is possible to use a limiting member that creates limitation states of at least two patterns so that adjacent ring images among the multiple ring images received by the light receiving element are not received by the light receiving element.

For example, the second limiting member may include a first ring limiting member (for example, mask member 135) and conversely a second ring limiting member (for example, mask member 136). The first ring limiting member (for example, mask member 135) has a light transmitting region that causes odd-numbered ring images from the inside among the multiple ring images to be received by the light receiving element, and a light shielding region that prevents even-numbered ring images from the inside from being received by the light receiving element. The second ring limiting member (for example, mask member 136) has a light transmitting region that causes even-numbered ring images from the inside among the multiple ring images to be received by the light receiving element, and a light shielding region that prevents odd-numbered ring images from the inside from being received by the light receiving element. That is, the second limiting member including the first ring limiting member and the second ring limiting member may be configured by multiple limiting members that limit to different ring images by skipping one ring image that is received by the light receiving element. Furthermore, the second limiting member may be configured by multiple limiting members that sequentially limit to different ring images by skipping two or more ring images that are received by the light receiving element.

Additionally, for example, the second limiting member may be a single limiting member which is equally divided in the circumferential direction of one circle corresponding to the multiple ring images received by the light receiving element, and light shielding regions are formed corresponding to the equally divided regions to shield light so that at least adjacent ring images are not received by the light receiving element. In this case, the limiting part may have a rotation part (for example, mask drive part 130A) that rotates the second limiting member around the center of the limiting member corresponding to the ring images. Then, the rotation part is controlled by the limiting controller, and as the second limiting member is rotated based on the angle of equal division, the limitation state of the ring images created by the second limiting member is sequentially changed.

Further, for example, the second limiting member may be a single limiting member which is equally divided into an even number of two or more (for example, two, four, six, etc.) in the circumferential direction of one circle corresponding to the multiple ring images received by the light receiving element, and for the equally divided regions, a first region and a second region are alternately arranged. The first region causes even-numbered ring images from the inside to be received by the light receiving element, and shields light so that odd-numbered ring images from the inside are not received by the light receiving element. The second region causes odd-numbered ring images from the inside to be received by the light receiving element, and shields light so that even-numbered ring images from the inside are not received by the light receiving element. In this case, as the second limiting member is rotated by the angle of equal division, the limitation state of the ring images created by the limiting member is changed to two patterns.

In addition, for example, the second limiting member may be a single limiting member which is equally divided into three or more parts in the circumferential direction of one circle corresponding to the multiple ring images, and corresponding to the number of divisions, light transmitting regions and light shielding regions are formed in each divided region so that adjacent ring images are not received by the light receiving element. In this case, as the second limiting member is sequentially rotated by the angle of equal division, the limitation state of the ring images created by the limiting member is sequentially changed.

For example, for the third limiting member, it is possible to use a limiting member with at least two patterns that divides the ring width of the ring images received by the light receiving element into at least two parts.

### <Limiting Controller>

For example, the limiting controller controls the limiting part to change the measurement region on the eye to be examined, which changes due to the limiting member, in at least two patterns. Thereby, more accurate and more appropriate measurement results can be obtained.

For example, the limiting controller may perform a first control to change the measurement region in at least two patterns by limiting the ring images received by the light receiving element to different diameters, with different limiting members. For example, the limiting controller may perform a second control to change the measurement region in at least two patterns by changing, with different limiting members, the limitation state of the ring images in at least two patterns so that adjacent ring images among the multiple ring images received by the light receiving element are not received by the light receiving element. For example, the limiting controller may perform a third control to change the measurement region in at least two patterns by dividing the ring width of the ring images received by the light receiving element into at least two parts, with different limiting members. For example, the limiting controller may perform at least one of the first control, the second control, and the third control. For example, the limiting controller may change the measurement region in at least two patterns by combining at least two of the first control, the second control, and the third control.

For example, the limiting controller may control the limiting part to reduce the number of ring images that can be received on the light receiving element, among the multiple ring images received on the light receiving element, during premeasurement. For example, the limiting controller may control the limiting part to limit the number of ring images that can be received on the light receiving element, among three or more ring images, to one or two during premeasurement. Then, for example, the limiting controller may control the limiting part so that the measurement region on the eye to be examined changes during main measurement after premeasurement, compared to during premeasurement. For example, the limiting controller may control the limiting part to increase the number of ring images that can be received on the light receiving element and change the measurement region on the eye to be examined in at least two patterns during main measurement, compared to during premeasurement. Thereby, appropriate measurement can be performed according to the condition of the eye to be examined during premeasurement and main measurement. For example, the limiting controller may control the limiting part to limit to a measurement region that is at least partially different from the measurement region on the eye to be examined, which is limited during premeasurement, during main measurement. Thereby, appropriate measurement can be performed according to the condition of the eye to be examined during premeasurement and main measurement.

For example, in a case where the limiting member is configured by the limiting part to be changeable to limitation states of at least two patterns so that adjacent ring images among the multiple ring images received by the light receiving element are not received by the light receiving element, the limiting controller may sequentially change at least two limitation states.

For example, in a case where the limiting member is configured by the limiting part to be changeable to a limitation state in which the ring width of the measurement region on the eye to be examined corresponding to each ring image received on the light receiving element is divided into at least two parts, the limiting controller may sequentially change the limitation state divided into at least two parts.

For example, in a case where any ring image received by the light receiving element can be arbitrarily selected by the limiting part among the multiple ring images that can be received on the light receiving element, the limiting controller may control the limiting part so that the preselected ring image is received by the light receiving element. Thereby, more appropriate eye refractive power information can be obtained according to the purpose.

### <Diopter Corrector>

For example, the diopter corrector adjusts the imaging state of the measurement index images received by the light receiving element according to the diopter of the eye to be examined. For example, the diopter corrector adjusts the imaging state of the measurement index images received by the light receiving element by moving at least the light receiving element in a measurement optical axis direction based on the eye refractive power information obtained from premeasurement of the eye refractive power.

### <Measurement Controller>

For example, the measurement controller performs a premeasurement to obtain the diopter of the eye to be examined in order to operate the diopter corrector, and performs a main measurement by operating the diopter corrector based on the measurement result obtained by the premeasurement.

### <Mode Switching Part>

For example, the mode switching part may be provided to enable switching between a first mode and a second mode. In the first mode, measurement is performed without using the limiting part during main measurement. In the second mode, measurement is performed using the limiting part during at least one of premeasurement and main measurement. For example, in the first mode, measurement can be performed speedily without imposing a burden on the eye to be examined, and in the second mode, more accurate and more appropriate measurement results can be obtained according to the condition of the eye to be examined.

Furthermore, for example, the mode switching part may be switched between a standard measurement mode of eye refractive power distribution in which the limiting part is not used in main measurement, and a special measurement mode in which the limiting part is used. In addition, for example, the special measurement mode may include at least one of a cataract eye measurement mode of eye refractive power distribution, a precision measurement mode of eye refractive power distribution, and a measurement region limiting measurement mode.

### <First Processor>

For example, the first processor processes the measurement index images (for example, ring images) received by the light receiving element to obtain eye refractive power information. For example, in a case where the conversion member causes multiple ring images to be received on the light receiving element as the measurement index images, the limiting member may be configured by the limiting part to be changeable to limitation states of at least two patterns so that adjacent ring images among the multiple ring images received by the light receiving element are not received by the light receiving element. Then, in a case where at least two limitation states are sequentially changed by the limiting controller, the first processor may obtain eye refractive power information from the multiple ring images by combining the measurement results of eye refractive power obtained by respectively processing the ring images sequentially received by the light receiving element. As a result, even in a case where the contrast of the ring images received by the light receiving element deteriorates due to scattering reflection in the eye such as in a cataract eye, the ring images are received with the interval between ring images widened, so it is easy to distinguish the ring images, and the eye refractive power information for each ring image can be obtained more accurately and more appropriately.

For example, in a case where the limiting member is configured by the limiting part to be changeable to a limitation state in which the ring width of the measurement region on the eye to be examined corresponding to each ring image received on the light receiving element is divided into at least two parts, and the limitation state divided into at least two parts is sequentially changed by the limiting controller, the first processor may obtain eye refractive power information from the multiple ring images by combining the measurement results of eye refractive power obtained by respectively processing the ring images sequentially received by the light receiving element due to the sequential change of the limitation state. As a result, even in a case where the ring width of the conversion member such as a ring lens cannot be manufactured finely, eye refractive power information in a finer measurement region can be obtained.

For example, in a case where an alignment deviation of the measurement optical system with respect to the eye to be examined is detected by the alignment detection part before and after a change in the measurement region on the eye to be examined caused by the limiting member, the first processor may correct the measurement region where the measurement result of eye refractive power has been obtained based on the displacement of the detected alignment deviation. For example, when the limitation state is changed by the limiting part and the first processor combines the measurement results of eye refractive power obtained by respectively processing the ring images sequentially received by the light receiving element, the first processor may correct the measurement region on the pupil corresponding to the ring image based on the displacement of the detected alignment deviation. As a result, even in a case where there is an alignment deviation, the measurement result of the measurement region corresponding to the eye refractive power information can be obtained more appropriately.

### <Mark Formation Part>

For example, the mark formation part in the second aspect forms a mark in the measurement index image to distinguish from which position of the measurement region on the eye to be examined, to which the measurement index image received by the light receiving element corresponds, the light beam comes from. For example, the mark formation part may be arranged in the vicinity of the conversion member or at a position conjugate to the conversion member, or may be directly provided on the conversion member. As a result, even in a case where the measurement index image on the light receiving element is disturbed or the signal level is low due to the condition of the eye to be examined, it is easy to distinguish which circle (what number) from the center position of the measurement optical axis the measurement index image belongs to, and more appropriate measurement results can be obtained.

For example, in a case where measurement index images composed of at least three circles of ring images centered on the optical axis of the measurement optical system are received by the light receiving element through the conversion member, the mark formation part may form different marks between adjacent ring images in at least one of the measurement index images. For example, in a case where measurement index images composed of a large number of point images arranged in at least three circles centered on the optical axis of the measurement optical system are received by the light receiving element through the conversion member, the mark formation part may form different marks between a large number of point images of adjacent circular arrangements in at least one of the measurement index images.

For example, in a case where multiple ring images are converted on the light receiving element by the conversion member, the mark formation part may form a gap in the ring image as a mark to distinguish at least one adjacent ring image. The mark formation part is not limited to a gap, but may be anything that serves as a mark for distinguishing from which position of the measurement region on the eye to be examined, to which the ring image corresponds, the light beam comes from. For example, in a case where the conversion member is configured by an optical member that converts ring images, the optical member itself may be modified to form marks in the ring images.

### <Second Processor>

For example, the second processor processes the measurement index image received by the light receiving element to obtain eye refractive power information. For example, the second processor may obtain the eye refractive power information of the missing portion of the measurement index image caused by the mark formed by the mark formation part by interpolation based on the measurement index image surrounding the missing portion. Thereby, more appropriate measurement results can be obtained.

### <Insertion/Removal Part, Insertion/Removal Controller>

For example, the insertion/removal part inserts and removes the mark formation part into and from the optical path. By inserting the mark formation part into the optical path when necessary, such as when measurement index images overlap, it becomes possible to distinguish the measurement index images received by the light receiving element. Further, in a case where the measurement index images received by the light receiving element can be distinguished, the mark formation part can be removed from the optical path to obtain measurement results more accurately with no missing part in the measurement index images.

For example, the insertion/removal controller may determine whether to insert the mark formation part into the optical path based on the reception state of the measurement index images received by the light receiving element, and control the driving of the insertion/removal part based on the determination result. The insertion/removal part may also be operated manually. Additionally, an operating part for selectively operating the insertion/removal part may be included.

### <Anterior Segment Image Acquisition Part, Third Processor>

For example, the anterior segment image acquisition part acquires an anterior segment image including the pupil of the eye to be examined. For example, the third processor processes the measurement index image received by the light receiving element and obtains eye refractive power information in the measurement region where the measurement index image corresponds on the pupil. For example, the third processor detects the vignetting state due to the pupil edge of the measurement light beam passing through the measurement region by comparing the pupil region in the anterior segment image with the corresponding measurement region on the pupil, and corrects the measurement region that is the target for obtaining eye refractive power information based on the detection result. Thereby, more appropriate measurement results can be obtained.

For example, the third processor may obtain eye refractive power information in the measurement region based on the reception result (for example, reception position) of the measurement index image received by the light receiving element regardless of the presence or absence of vignetting of the measurement light beam passing through the measurement region, and with respect to the eye refractive power information of a first measurement region where vignetting of the measurement light beam is detected, the third processor may replace it with a second measurement region that corrects the first measurement region based on the vignetting state of the measurement light beam passing through the first measurement region. In a case where the conversion member converts multiple ring images, the third processor may, with respect to the first measurement region where vignetting of the measurement light beam is detected among multiple measurement regions, replace it with a second measurement region that corrects the first measurement region based on the vignetting state of the measurement light beam.

For example, the third processor may obtain eye refractive power information for each meridian direction with reference to the measurement optical axis of the measurement optical system, and may determine the second measurement region by a remaining region width obtained by subtracting the vignetting portion of the measurement light beam from the first measurement region for each meridian direction. As a result, even in a case where the pupil edge is decentered with respect to the measurement optical axis, more accurate and more appropriate measurement results can be obtained.

For example, the conversion member may be a member that causes ring images to be received on the light receiving element as measurement index images. In this case, the third processor may obtain eye refractive power information for each meridian direction based on the measurement optical axis of the measurement optical system based on the ring images, and may correct the measurement region for each meridian direction where eye refractive power information has been obtained based on the detection result of the vignetting state of the measurement light beam passing through the measurement region. Thereby, more appropriate measurement results can be obtained for each meridian direction.

For example, in a case where the conversion member causes multiple ring images to be received on the light receiving element as measurement index images, the third processor may, when it is detected that a part of the measurement light beam passing through one ring-shaped measurement region among the ring-shaped measurement regions corresponding to the ring images has vignetting due to the pupil edge, correct the measurement region for obtaining eye refractive power information to a measurement region with a narrowed width of the one ring-shaped measurement region based on the vignetting state of the measurement light beam.

For example, in a case where the conversion member causes ring images to be received on the light receiving element as measurement index images, the third processor may further obtain eye refractive power information in the measurement region corresponding to the ring image received on the light receiving element on the pupil, based on the detection result of the vignetting state of the measurement light beam due to the pupil edge and the reception position of the ring image received on the light receiving element due to the measurement light beam passing through the pupil. For example, the third processor may obtain eye refractive power information corresponding to the center position of the ring width of the ring image in a case where there is no vignetting of the measurement light beam based on the detection result of the vignetting state of the measurement light beam and the reception position of the ring image. Alternatively, the third processor may obtain eye refractive power information based on a function or table in which a correspondence relationship between the vignetting state of the measurement light beam and the eye refractive power with respect to the position of the ring image is predetermined. As a result, even in a case where the focal point of the ring image formed by the conversion member is not positioned on the light receiving element and the ring image is blurred, there is information on the vignetting state of the measurement light beam, making it possible to obtain more appropriate eye refractive power.

### First Embodiment

An example of the ophthalmic measurement device in the first embodiment will be described.

### Overall Configuration

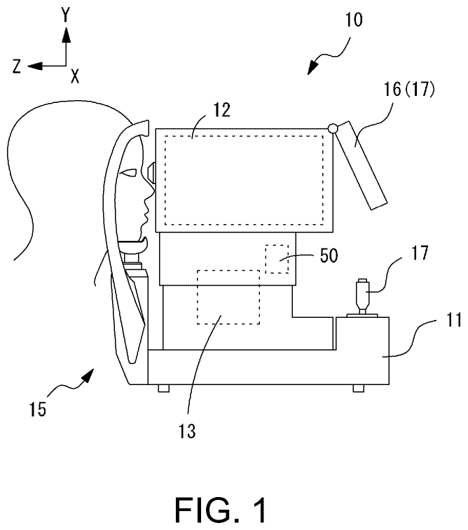

FIG. 1 is a diagram of the external appearance of the ophthalmic measurement device 10. The ophthalmic measurement device 10 in FIG. 1 is a stationary type, but is not necessarily limited thereto, and may also be a handheld type.

The ophthalmic measurement device 10 of the disclosure includes at least a base 11, a measurement part 12, an alignment drive part 13, a face support part 15, a monitor 16, an operation part 17, and a control part 50.

The measurement part 12 houses optical systems, etc. used for measuring an eye to be examined. The face support part 15 is fixed to the base 11 and fixes the eye to be examined by supporting the face of the examinee. The alignment drive part 13 changes the positional relationship between the measurement part 12 and the eye to be examined. For example, the alignment drive part 13 moves the measurement part 12 three-dimensionally with respect to the base 11, thereby moving the measurement part 12 in an X direction (left-right direction), a Y direction (up-down direction), and a Z direction (front-back direction) with respect to the eye to be examined.

The monitor 16 displays various information (for example, an anterior segment image of the eye to be examined, measurement results of optical characteristics of the eye to be examined, etc.). The operation part 17 performs various settings. The monitor 16 may function as a touch panel, and the monitor 16 may also serve as the operation part 17.

### <Optical System>

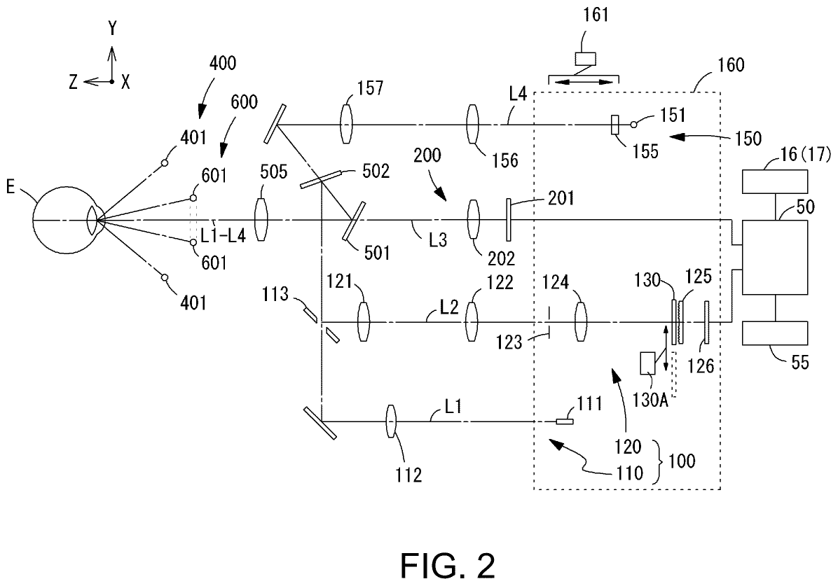

FIG. 2 is a schematic diagram of the optical systems of the ophthalmic measurement device 10. As an example, the measurement part 12 includes a measurement optical system 100, a fixation target presentation optical system 150, an observation optical system 200, an index projection optical system 400, an alignment index projection optical system 600, etc. The measurement part 12 also includes half mirrors 501 and 502 that branch and combine the optical paths of the optical systems, an objective lens 505, etc.

### <Measurement Optical System>

The measurement optical system 100 is used to objectively measure the eye refractive power of the eye to be examined. The measurement optical system 100 has a light projecting optical system 110 and a light receiving optical system 120. The light projecting optical system 110 projects a spot-shaped measurement light beam onto the fundus of the eye to be examined through the center portion of the pupil of the eye to be examined. The light receiving optical system 120 extracts the reflected light beam of the measurement light beam reflected by the fundus in multiple ring shapes or multiple spot shapes through the pupil.

The light projecting optical system 110 includes a light source 111, a relay lens 112, a hole mirror 113, an objective lens 505, etc. The light source 111 is arranged on an optical axis L1 and is arranged at a fundus conjugate position. The light source 111 may be an SLD (Superluminescent diode) light source, an LED (Light Emitting Diode) light source, or any other light source. In this example, the light source 111 is an infrared light source and may project an infrared light beam as the measurement light beam. For example, the measurement light beam may be a near-infrared light beam with a peak between wavelengths of 800 nm to 900 nm. As an example, the measurement light beam may be a near-infrared light beam with a peak at wavelength of 870 nm. The opening of the hole mirror 113 is arranged at the pupil conjugate position. The measurement light emitted from the light source 111 is projected onto the fundus of the eye to be examined through the relay lens 112, the hole mirror 113, the half mirrors 501 and 502, and the objective lens 505.

The light receiving optical system 120 includes an objective lens 505, a hole mirror 113, relay lenses 121 and 122, a light receiving aperture 123, a collimator lens 124, a ring lens 125 as an example of the conversion member, an imaging element 126, etc. Furthermore, the light receiving optical system 120 includes a mask 130 as an example of the limiting member. The ring lens 125 has a function to convert the reflected light from the fundus into multiple (divided into multiple in the meridian direction) ring images on the imaging element 126 for light reception (imaging). The ring lens 125 is arranged at a conjugate position of the pupil (pupil opening) of the eye to be examined. The light receiving aperture 123 is arranged at the fundus conjugate position. The imaging element 126 is arranged at the fundus conjugate position. The mask 130 is arranged at a conjugate position of the ring lens 125 or in the vicinity of the ring lens 125. In FIG. 2, the mask 130 is arranged in the vicinity of the ring lens 125 as an example.

In the disclosure, the conjugate position also includes an approximate conjugate position. “Approximate conjugate” means that the position does not need to be perfectly conjugate, and may be conjugate with the accuracy required in relation to measurement precision. In addition, “vicinity” means that the position may be near with the accuracy required in relation to measurement precision.

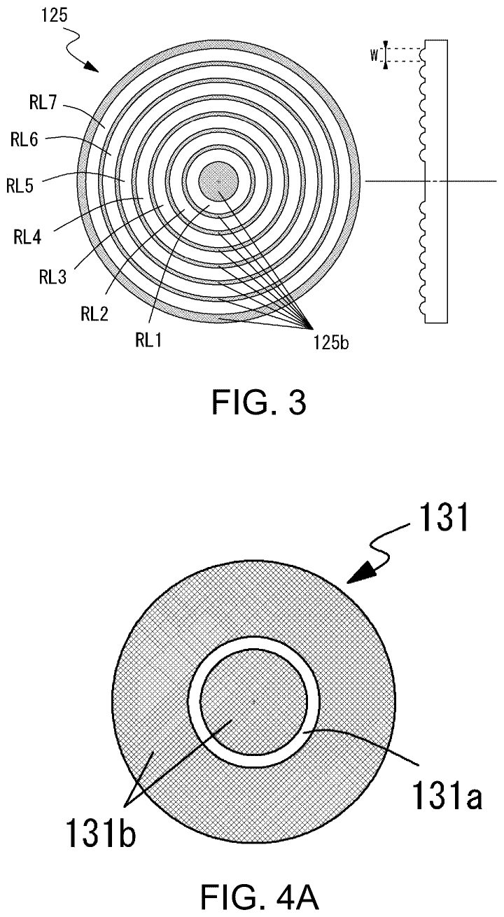

FIG. 3 is a diagram showing an example of the ring lens 125 of the disclosure. The left side in FIG. 3 shows a plan view of the ring lens 125, and the right side shows a cross-sectional view of the ring lens 125. For example, the ring lens 125 includes ring-shaped columnar lens portions and is configured to form three or more ring images on the imaging element 126. In the example of FIG. 3, ring-shaped lens portions RL1, RL2, RL3, RL4, RL5, RL6, and RL7 are provided from the inside to form seven ring images. For example, the diameter D1 (diameter of the center portion of the lens portion) of the innermost lens portion RL1 on the pupil is made as small as possible with a size that minimizes the influence of corneal reflected light of the measurement light projected by the light projecting optical system 110. For example, the diameter D1 on the pupil is set to 1.5 mm or less. For example, the diameter D7 (diameter of the center portion of the lens portion) of the outermost lens portion RL7 on the pupil is sized to cover an anticipated large pupil diameter of the human eye. For example, the diameter D7 on the pupil is set to 6 mm or more. Further, in the example, the lens portions RL1 to RL7 are formed with the same ring width W. Then, each ring region on the pupil corresponding to each ring region of the lens portions RL1 to RL7 is set as a measurement region.

In the ring lens 125, light shielding portions 125b are formed in the inner region of the lens portion RL1, in the regions between the lens portions RL1 to RL7, and in the outer region of the lens portion RL7. The gaps between adjacent lens portions are preferably made minute with the same interval. The number of rings in the ring lens 125 is not limited thereto, as long as there are three or more.

In this example, the mask 130 has multiple mask members as limiting members prepared to selectively limit some of the multiple ring images received on the imaging element 126 by the ring lens 125. The mask 130 is selectively inserted into and removed from the optical path by a mask drive part 130A. For example, the multiple mask members included in the mask 130 are arranged on a turret plate (not shown), and are selectively placed in the optical path by rotating the turret plate with the mask drive part 130A. Also, a light transmitting opening is provided in the turret plate, and by inserting this opening into the optical path, a state in which the mask member is removed from the optical path is created. For example, the mask member may be a transparent material such as glass with a light shielding coating applied, or a sheet metal with physical holes may be used. In addition, the mask 130 may be an element that can electronically control the mask state, such as a transmissive LCD. In this case, light transmitting regions and shielding regions can be freely created.

The measurement light beam projected onto the fundus of the eye to be examined by the light projecting optical system 110 is reflected at the fundus. The reflected light beam from the fundus passes through the objective lens 505 and the half mirrors 501 and 502, is reflected by the mirror portion of the hole mirror 113 to proceed in the direction of an optical axis L2, and reaches the imaging element 126 through the relay lenses 121 and 122, the light receiving aperture 123, the collimator lens 124, the mask 130, and the ring lens 125. As a result, the imaging element 126 is able to receive seven ring images as measurement index images converted by the ring lens 125.

The measurement optical system 100 may be any optical system having a light projecting optical system that projects a measurement light beam onto the fundus of the eye to be examined and a light receiving optical system that receives the reflected light beam of the measurement light beam reflected by the fundus, and is not limited to the above configuration. For example, the measurement optical system 100 may be an optical system that projects a spot index onto the fundus and detects the reflected light beam of the spot index at the fundus with a Shack-Hartmann sensor. In this case, a Hartmann plate may be arranged in place of the ring lens 125. The reflected light beam of the spot index is converted by being separated into multiple light beams by the Hartmann plate, and multiple spot-like measurement index images are detected as a large number of point images are captured on the imaging element 126.

### <Fixation Target Presentation Optical System>

The fixation target presentation optical system 150 presents a fixation target to the eye to be examined. The fixation target presentation optical system 150 is used to fixate the eye to be examined, and is also used to provide fogging and accommodation load to the eye to be examined.

The fixation target presentation optical system 150 includes a light source 151, a fixation target plate 155, lenses 156 and 157, an objective lens 505, etc. The light source 151 is arranged on an optical axis L5. The fixation target plate 155 is arranged at the fundus conjugate position. A fixation light beam from the light source 151 passes through the fixation target plate 155, the lens 156, and the lens 157, then passes through the half mirror 502 to become coaxial with the optical axis L1, is reflected by the half mirror 501, and further reaches the fundus through the objective lens 505.

### <Observation Optical System>

The observation optical system 200 is used to acquire an observation image of the anterior segment of the eye to be examined. The observation image is used for alignment, analysis of the measurement region on the pupil corresponding to the ring lens 125, etc. For example, the observation optical system 200 includes an imaging element 201, a lens 202, an objective lens 505, etc. The imaging element 205 is arranged at the pupil conjugate position. The observation optical system 200 also serves as a detection optical system that detects index images projected onto the cornea from the index projection optical system 400 and index images projected onto the cornea from the alignment index projection optical system 600.

### <Index Projection Optical System>

The index projection optical system 400 is used to measure the corneal shape. The index projection optical system 400 projects indices for measuring the corneal shape onto the anterior segment from the front facing the eye to be examined. The index projection optical system 400 includes multiple point light sources 401. The point light sources 401 project infinity indices by irradiating parallel light beams onto the cornea. The point light sources 401 are arranged symmetrically up and down and left and right with the optical axis L1 as the center. For instance, in this example, two point light sources are provided on each of the left and right sides. As a result, four point image indices are projected onto the cornea. Nevertheless, the number of indices is not limited thereto, and there may be three or more point image indices. Also, the shape of the indices is not limited thereto, and line-shaped indices may be used.

### <Alignment Index Projection Optical System>

The alignment index projection optical system 600 is used to align (position) the measurement part 12 with respect to the eye to be examined. The alignment index projection optical system 600 includes an alignment light source 601. The alignment light source 601 projects a finite distance index by irradiating a diffused light beam onto the cornea. The alignment light source 601 is arranged in a ring shape with the optical axis L1 as the center. As a result, a ring index (so-called Mayer ring image) is projected onto the cornea.

In this example, the alignment index projection optical system is formed by the alignment light source 601 and the index projection optical system 400. For example, operating distance adjustment may be performed by moving the measurement part 12 in the front-back direction so that the Purkinje image of the alignment light source 601 and the Purkinje image of the index projection optical system 400 are captured at a predetermined ratio.

### <Diopter Corrector>

In this example, the light source 111, the ring lens 125, and the imaging element 126 in the measurement optical system 100, and the light source 151 and the fixation target plate 155 in the fixation target presentation optical system 150 can be integrally moved along the optical axis as a driver 160. For example, the presentation distance of the fixation target plate 155 to the eye to be examined (that is, the presentation position of the fixation target) can be changed by moving the driver 160 through the drive part 161 according to the eye refractive power of the eye to be examined (that is, the diopter of the eye to be examined). Then, the imaging state (focusing state) of the ring image received on the imaging element 126 is adjusted according to the diopter of the eye to be examined by integrally moving the ring lens 125 and the imaging element 126 of the light receiving optical system 120 in the optical axis direction so that the imaging element 126 is near a position conjugate to the fundus of the eye to be examined.

### <Control System>

In FIG. 2, the control part 50 includes a CPU (processor), a RAM, a ROM, etc. The CPU controls the driving of each part in the ophthalmic measurement device 10. The RAM temporarily stores various information. The ROM stores various programs to be executed by the CPU. The control part may be configured by multiple control parts (that is, multiple processors).

The control part 50 is electrically connected to the alignment drive part 13, the monitor 16, a non-volatile memory 55 (hereinafter, memory 55), etc. In addition, the control part 50 is electrically connected to each light source, each imaging element, each drive part, etc. of the measurement part 12. The memory 55 is a non-transitory storage medium that can retain the stored content even when power supply is cut off. For example, a hard disk drive, a flash ROM, a USB memory, etc. can be used as the memory 55. The measurement results, etc. of the eye to be examined may be stored in the memory 55.

The control part 50 functions as a processor that processes the anterior segment image obtained by the imaging element 201 to obtain various information. The control part 50 also functions as a processor that processes the output image from the imaging element 126, or the addition image of data sequentially output from the imaging element 126, to obtain eye refractive power information. Further, the control part 50 controls the driving of the mask drive part 130A and changes the measurement region on the eye to be examined, which is changed by the mask member included in the mask 130, in at least two patterns.

### Operation

The operation of the ophthalmic measurement device 10 having the above configuration will be described. In this example, the ophthalmic measurement device 10 performs preliminary measurement before main measurement, and main measurement after preliminary measurement. For the main measurement, a standard measurement mode of eye refractive power distribution in which the mask 130 is not used, and a special measurement mode in which the mask 130 is used, are prepared. For example, in the special measurement mode, a cataract eye measurement mode of eye refractive power distribution, a precision measurement mode of eye refractive power distribution, and a measurement region limiting measurement mode are prepared. Each measurement mode can be switched by the examiner operating the operation part 17, and the ophthalmic measurement device 10 (control part 50) can also automatically switch between these modes. Details of each measurement mode will be described later.

### <Alignment>

The examiner instructs the examinee to place his/her face on the face support part 15. The control part 50 executes alignment of the measurement part 12 with respect to the eye to be examined based on signals from the operation part 17. For example, the control part 50 turns on the light source 151 of the fixation target presentation optical system 150 to present a fixation target to the eye to be examined. Further, the control part 50 turns on each light source of the index projection optical system 400 and the alignment index projection optical system 600 to project alignment indices onto the cornea of the eye to be examined. For example, the control part 50 detects alignment index images (point images and Myer ring images) from the observation image captured by the imaging element 201 of the observation optical system 200, and detects the alignment state of the measurement part 12 in the X direction, the Y direction, and the Z direction with respect to the eye to be examined. Then, the control part 50 controls the driving of the alignment drive part 13 based on the detection result of the alignment state, and adjusts the positional relationship of the measurement part 12 with respect to the eye to be examined to a predetermined positional relationship.

### <Preliminary Measurement>

After alignment is completed, preliminary measurement is executed before the main measurement to obtain the eye refractive power distribution. The preliminary measurement is primarily performed to obtain diopter correction information of the eye to be examined in order to position the imaging element 126 near a position conjugate to the fundus of the eye to be examined for the main measurement. The preliminary measurement is also performed to apply fogging to the eye to be examined during the main measurement.

In the initial stage of the preliminary measurement, the imaging element 126 is placed at a position where the eye to be examined is at OD (a power where there is no refractive error in the eye to be examined, where “D” indicates diopter, the unit of power). Here, in a case where the spherical power of the refractive error of the eye to be examined is low (for example, 3D or less in both plus and minus), the seven ring images received on the imaging element 126 through the ring lens 125 are separated from each other, and the contrast does not significantly decrease, allowing each of the seven ring images to be distinguished and detected. On the other hand, in a case where the spherical power of the refractive error of the eye to be examined is high on the minus side (for example, 7D or more), the seven ring images are reduced, and adjacent ring images may approach each other, making it difficult to distinguish and detect each ring image. Also, in a case where the spherical power of the refractive error of the eye to be examined is high on the plus side (for example, 7D or more), the ring images expand and blur, causing the contrast of the ring images to decrease and making it difficult to distinguish adjacent ring images. Additionally, depending on the condition of the eye to be examined, such as typically in cataract eyes, the state of reflection and scattering in the eye differs, which may make it more difficult to distinguish adjacent ring images.

Therefore, in the preliminary measurement, the number of ring images that can be received on the imaging element 126 is reduced from the multiple (seven in this example) ring images. For example, the mask member of the mask 130 is inserted into the optical path so that the number of ring images received on the imaging element 126 is limited to one or two. Whether to use a measurement mode using the mask 130 in the preliminary measurement stage or a measurement mode without a mask may be automatically switched by the control part 50. For example, initially, images of seven ring images received on the imaging element 126 in a state without the mask 130 are processed, and if it is determined that it is difficult to distinguish each ring image, the control part 50 automatically switches to a measurement mode using the mask 130. Regarding the measurement mode without a mask, the center portion of the ring lens 125 may have a mask for removing corneal reflection bright points.

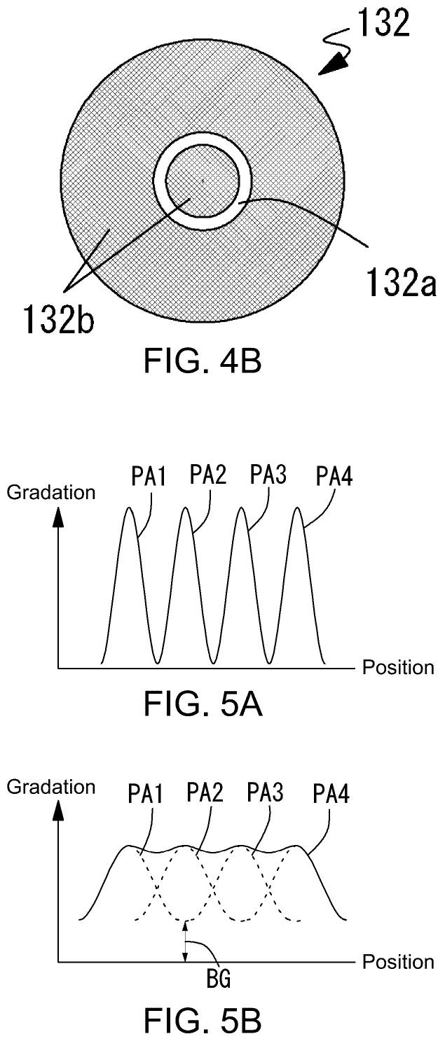

FIG. 4A and FIG. 4B are examples of the mask members used in the preliminary measurement stage. The mask member 131 in FIG. 4A has a ring-shaped light transmitting region 131a formed so that the ring image received on the imaging element 126 is limited to one ring image from the lens portion RL3 shown in FIG. 3, and other regions are light shielding regions 131b. For example, the diameter D of the center of the ring width of the light transmitting region 131a on the pupil is 3.2 mm, the same as the lens portion RL3. Also, the ring width of the light transmitting region 131a is the same width W as the lens portion RL3.

When the mask member 131 of the mask 130 is placed on the optical path, the ring image received on the imaging element 126 is limited to one ring image from the lens portion RL3. Thus, even if the spherical power of the refractive error of the eye to be examined is high, the control part 50 analyzes the ring image captured by the imaging element 126 to determine the eye refractive power in each meridian direction, and by applying predetermined processing to this eye refractive power, at least the spherical power (here, the spherical power in the preliminary measurement) is obtained. The control part 50 may also obtain the astigmatic power along with the spherical power in the preliminary measurement.

The mask member 131 is applicable to cases where the pupil diameter of the eye to be examined is of standard size (for example, 3.2 mm or more). In a case where the pupil diameter of the eye to be examined is smaller than the standard size, a mask member may be prepared to limit the ring image to the ring image from the lens portion RL2 or RL1 which is inside the lens portion RL3. For example, the mask member 132 in FIG. 4B is an example in which a ring-shaped light transmitting region 132a is formed so that the ring image received on the imaging element 126 is limited to correspond to the lens portion RL2 shown in FIG. 3. Regions other than the light transmitting region 132a are light shielding regions 132b. The diameter D of the center of the ring width of the light transmitting region 132a on the pupil is, for example, 2.3 mm. The width of the light transmitting region 132a is the same width W as the lens portion RL2.

For example, in a case where multiple mask members (in this example, mask member 131 and mask member 132) are prepared for use in the stage of preliminary measurement, the control part 50 may determine which one to use according to the pupil size of the eye to be examined, as follows. The control part 50 performs image processing on the anterior segment image acquired by the imaging element 201 of the observation optical system 200 and determines the size of the pupil region of the eye to be examined. The control part 50 determines to use the mask member 131 in a case where the size of the pupil region is larger than the measurement region of the lens portion RL3 on the pupil. The control part 50 determines to use the mask member 132 in a case where the size of the pupil region is smaller than the measurement region of the lens portion RL3 on the pupil (in other words, in a case of a small pupil diameter). Then, the control part 50 drives the mask drive part 130A based on the determination result and selectively switches between the mask members 131 and 132 in the mask 130.

In the stage of preliminary measurement, a mask member that limits the number of ring images received on the imaging element 126 to two may be used. For example, a mask member having two light transmitting regions, the light transmitting region 131a shown in FIG. 4A and the light transmitting region 132a shown in FIG. 4B, may be used. In this case, since only two ring images are received on the imaging element 126, distinction is relatively easy. Then, in a case where the eye to be examined has a small pupil diameter, the eye refractive power for the small pupil diameter can be obtained by receiving the ring image of the measurement light beam passing through the light transmitting region 132a on the imaging element 126.

### <Standard Measurement Mode of Eye Refractive Power Distribution>

After the preliminary measurement is completed, the process transitions to the main measurement. First, the standard measurement mode of eye refractive power distribution will be described. The standard measurement mode of eye refractive power distribution is a measurement mode that obtains the eye refractive power distribution over a wide range within the pupil of the eye to be examined without using the mask 130. For example, the measurement result of the eye refractive power distribution is used for corneal correction surgery, etc., which changes the corneal shape using laser light. The standard measurement mode of eye refractive power distribution is used for an eye to be examined in a standard state in which there is little influence of reflection or scattering in the eye.

If the spherical power of the refractive power of the eye to be examined is obtained by the preliminary measurement, the diopter corrector is activated. For example, based on the spherical power in the preliminary measurement, the fixation target plate 155, the ring lens 125, the imaging element 126, etc. included in the driver 160 are integrally moved along the optical axis by the driving of the drive part 161, so that the imaging element 126 is placed near a position conjugate to the fundus of the eye to be examined, and the fixation target plate 155 is placed at a fogging start position where the eye to be examined can focus. As a result, the eye to be examined can clearly observe the fixation target. After that, the fixation target plate 155 is moved away from the fogging start position, and a predetermined amount of diopter fogging is added. This releases the accommodation of the eye to be examined, and the measurement result of the eye refractive power approaches the true value.

In the main measurement, the mask member 131 or 132 of the mask 130 used in the preliminary measurement is removed from the optical path. Then, in the standard measurement mode of eye refractive power distribution, the eye refractive power distribution is measured in a state in which the multiple ring images received on the imaging element 126 are not limited by the mask 130. For example, the control part 50 continuously captures each ring image on the imaging element 226 at predetermined timing intervals. For example, the control part 50 also acquires images of ring images with reduced noise light by performing addition processing on each ring image. Then, for example, the control part 50 thins each ring image in the acquired image and determines the eye refractive power distribution for each meridian direction (for example, every 1 degree) for each ring image. For example, if seven ring images corresponding to the ring lens 125 shown in FIG. 3 are acquired, a detailed eye refractive power distribution for each meridian direction is determined for the positions of the diameter sizes on the pupil of the lens portions RL1 to RL7, with reference to the corneal center. The measurement result of the eye refractive power distribution is stored in the memory 55.

In addition, when the detailed eye refractive power distribution of the eye to be examined is determined, the measurement result is displayed on the screen of the monitor 16. For example, the measurement result of the eye refractive power distribution is displayed as a color map, a contour map, or the like that is color-coded according to the magnitude of the refractive power.

Since the mask 130 is not used in this standard measurement mode, measurement can be performed speedily without imposing a burden on the eye to be examined, compared to the cataract eye measurement mode and the precision measurement mode of eye refractive power distribution which will be described later.

### <Cataract Eye Measurement Mode of Eye Refractive Power Distribution>

In the measurement of eye refractive power distribution in the main measurement, depending on the condition of the eye to be examined, the state of reflection and scattering in the eye differs, which causes the profile of the ring images received by the imaging element 226 to change. Even if each ring image is processed by addition, it may still be difficult to distinguish adjacent ring images. For example, in a case where the eye to be examined is a cataract eye, the scattering component in the eye increases, making the contrast of ring images more likely to decrease and making it difficult to distinguish adjacent ring images. Therefore, during the main measurement after preliminary measurement, the driving of the mask 130 is controlled so that the measurement region on the eye to be examined changes, compared to during the preliminary measurement. In a case where the number of ring images that can be received by the imaging element 226 is reduced during the preliminary measurement, during the main measurement, the driving of the mask 130 is controlled to increase the number of ring images that can be received by the imaging element 226, compared to during the preliminary measurement.

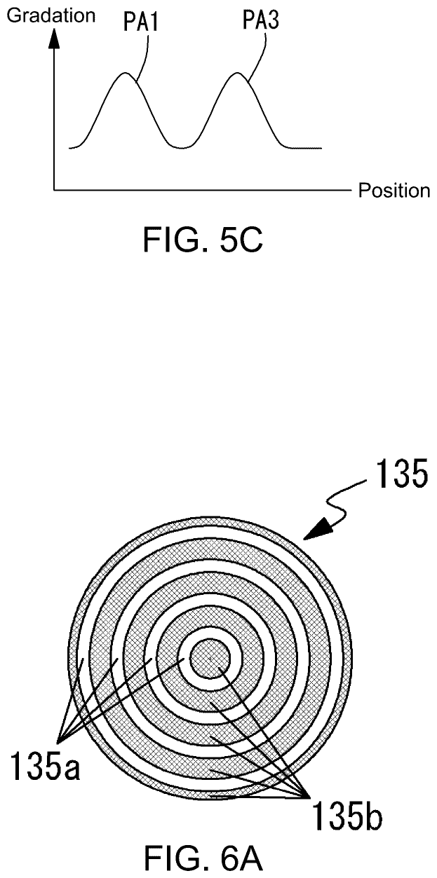

FIG. 5A, FIG. 5B, and FIG. 5C are diagrams explaining the distinction between adjacent ring images. FIG. 5A shows an example of the profiles (gradation with respect to position) of signals of four ring images PA1, PA2, PA3, and PA4 in an eye to be examined with little reflection and scattering in the eye. In this case, the four profiles have obtained sufficient contrast to distinguish the ring images PA1, PA2, PA3, and PA4.

On the other hand, FIG. 5B shows an example of the profiles of signals of four ring images PA1 to PA4 in an eye to be examined with a lot of reflection and scattering in the eye. Due to the different condition of reflection and scattering in the eye, the background BG of the ring image profile changes. In the case of the example of FIG. 5B, the gradation between adjacent ring images does not sufficiently drop, resulting in low contrast. Therefore, it is difficult to distinguish the positions of the four ring images PA1, PA2, PA3, and PA4.

Thus, the mask 130 is used to address this issue. FIG. 5C shows an example of the profiles of signals where the ring images PA2 and PA4 are limited so as not to be received by the imaging element 126, compared to the ring images PA1, PA2, PA3, and PA4 in FIG. 5B. In this case, the gradation between the ring image PA1 and the ring image PA3 obtains sufficient contrast to distinguish the two ring images. Accordingly, by placing the mask member of the mask 130 in the optical path to prevent adjacent ring images among the ring images received by the imaging element 126 through the ring lens 125 from being received, it is possible to distinguish ring images even in an eye to be examined with a lot of reflection and scattering in the eye. Then, by sequentially switching the mask member placed in the optical path, it is possible to distinguish other ring images.

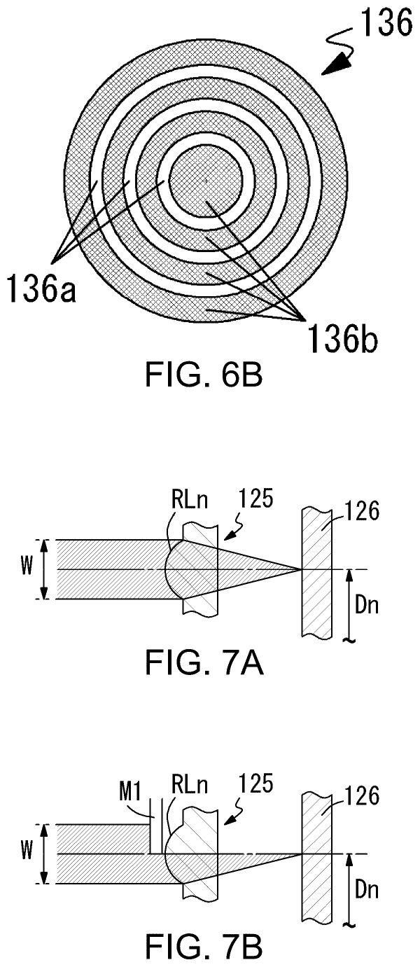

FIG. 6A and FIG. 6B are examples of mask members that limit adjacent ring images from being received in the mask 130. The mask member 135 in FIG. 6A has ring-shaped light shielding regions 135b and ring-shaped light transmitting regions 135a. The light shielding regions 135b are provided so that ring images from even-numbered lens portions RL2, RL4, and RL6 from the inside among the lens portions RL1 to RL7 are not received by the imaging element 126. The light transmitting regions 135a are formed so that ring images from odd-numbered lens portions RL1, RL3, RL5, and RL7 are received by the imaging element 126. The mask member 136 in FIG. 6B has the light shielding and transmitting regions reversed from those of the mask member 135. That is, the mask member 136 has ring-shaped light shielding regions 136b and ring-shaped light transmitting regions 136a. The light shielding regions 136b are formed so that ring images from odd-numbered lens portions RL1, RL3, RL5, and RL7 are not received by the imaging element 126. The light transmitting regions 136a are formed so that ring images from even-numbered lens portions RL2, RL4, and RL6 are received by the imaging element 126.

When executing the main measurement in the cataract eye measurement mode of eye refractive power distribution, for example, the mask member 135 is inserted into the optical path first, allowing ring images from odd-numbered lens portions RL1, RL3, RL5, and RL7 to be received by the imaging element 126. This first image of odd-numbered ring images is stored in the memory 55 (or memory held by the control part 50) and acquired. Next, the mask 130 inserted into the optical path is changed to the mask member 136, allowing ring images from even-numbered lens portions RL2, RL4, and RL6 to be received by the imaging element 126. This second image of even-numbered ring images is stored in the memory 55 (or memory held by the control part 50) and acquired. The control part 50 processes the acquired first image and determines the eye refractive power distribution for each meridian direction at the measurement positions of the odd-numbered ring images. Similarly, the control part 50 processes the acquired second image and determines the eye refractive power distribution for each meridian direction at the measurement positions of the even-numbered ring images. This makes it possible to distinguish adjacent ring images even for an eye to be examined with a lot of reflection and scattering in the eye, thereby obtaining more accurate eye refractive power. Finally, the control part 50 obtains the overall eye refractive power distribution based on the ring images from the lens portions RL1 to RL7 by combining the measurement results obtained by respective processing of the first image and the second image.

Although the above describes an example where the mask members 135 and 136 acquire ring images from the ring lens 125 by skipping one (every other one), the interval between adjacent ring images may be increased by skipping two (every third one) or more by increasing the number of mask members that are sequentially changed (switched). In this case, the control part 50 can still obtain the overall eye refractive power distribution from the ring lens 125 by sequentially changing the mask members and combining the measurement results of eye refractive power obtained by respective processing of the ring images sequentially received by the imaging element 126.

In the main measurement, whether to switch to the cataract eye measurement mode of eye refractive power distribution may be automatically determined (selected) by the control part 50 based on at least one of: the output signal of ring images from the imaging element 126 during preliminary measurement, and the output signal of ring images from the imaging element 126 at the start of main measurement in a state where the mask 130 is not being used. Alternatively, the switching to the cataract eye measurement mode of eye refractive power distribution may be done by the examiner by viewing and determining the state of ring images in the image received by the imaging element 126 and displayed on the monitor 16, and operating the operation part 17. Additionally, in a case where an anterior segment transillumination image has been acquired, the switching to the cataract eye measurement mode may be automatically determined by the control part 50 based on that anterior segment transillumination image.

### <Precision Measurement Mode of Eye Refractive Power Distribution>

In the eye refractive power distribution measurement, the number of divisions in the measurement region on the pupil of the eye to be examined is determined by the number of ring-shaped lens portions (RL1 to RL7 in the example) provided in the ring lens 125. When the number of ring-shaped lens portions is increased, the ring images received by the imaging element 126 become dense, making it difficult to distinguish adjacent ring images. Additionally, it is difficult to make a ring lens 125 with fine ring-shaped lens portions, and there is a limit.

Therefore, the following describes an example of effectively increasing the number of divisions in the measurement region of the eye to be examined to obtain a more precise eye refractive power distribution by devising the mask member 135 rather than increasing the number of ring-shaped lens portions in the ring lens 125. For example, in the precision measurement mode, a mask member is used that divides the ring width of the ring images received by the imaging element 126 into two parts or more.

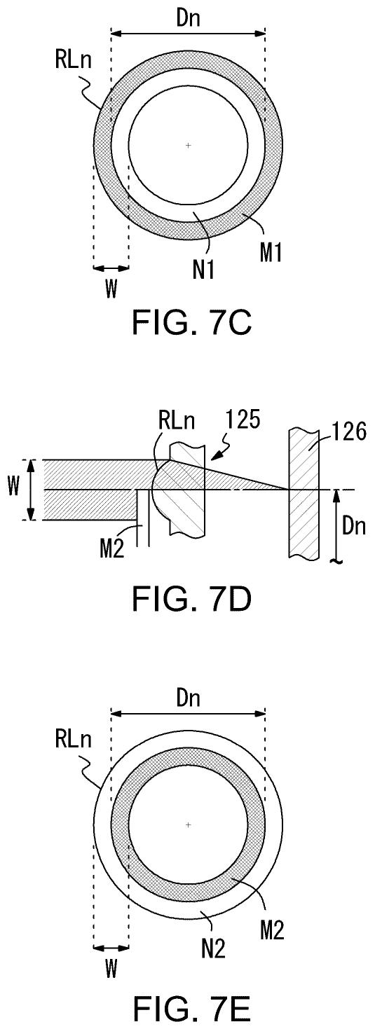

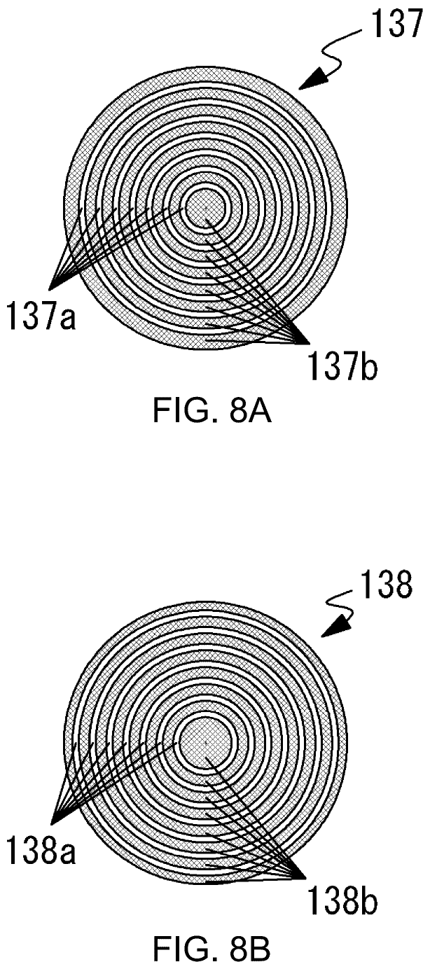

FIG. 7A to FIG. 7E are diagrams explaining the measurement of precise eye refractive power distribution. FIG. 7A shows the passage of the measurement light beam (reflected light from the fundus) and imaging on the imaging element 126 for a certain ring-shaped lens portion RLn of the ring lens 125. The ring width of the lens portion RLn on the pupil of the eye to be examined is W, and the diameter at the center of the ring width is Dn. For example, if the diameter Dn is 4.0 mm and the ring width W is 0.2 mm, the eye refractive power corresponding to the measurement region of 3.8 mm to 4.2 mm on the pupil of the eye to be examined is obtained with one lens portion RLn. For example, the measurement result in this case is expressed as a measurement result at a pupil diameter of 4.0 mm corresponding to the diameter Dn at the center of the ring width W.

FIG. 7B shows an example of a case where the ring width W of the ring-shaped lens portion RLn is divided into two parts. For example, by placing the mask member M1 in the vicinity of the lens portion RLn to shield the outer half region of the lens portion RLn, the light beam passing through the lens portion RLn is limited. In this case, the measurement region on the pupil of the eye to be examined becomes the region between the diameter Dn and the diameter (DN-W). That is, with the same dimensional example as in FIG. 7A, the measurement region on the pupil becomes 3.8 mm to 4.0 mm. The measurement result in this case is expressed as a measurement result at a pupil diameter of 3.9 mm corresponding to the center of the light beam passing through the lens portion RLn.

FIG. 7C is a diagram of the mask member M1 and the region of the lens portion RLn in FIG. 7B when viewed from the front. The mask member M1 has a pattern in which the inner half of the lens portion RLn is a transmitting region N1.

Next, compared to FIG. 7B, the light beam passing through the lens portion RLn is limited by shielding the inner half region of the lens portion RLn with the mask member M2 as shown in FIG. 7D. In this case, the measurement region on the pupil of the eye to be examined becomes the region between the diameter Dn and the diameter (DN+W/2). That is, with the same dimensional example as in FIG. 7A, the measurement region on the pupil is 4.0 mm to 4.2 mm. Then, the measurement result in this case is expressed as a measurement result at a pupil diameter of 4.1 mm corresponding to the center of the light beam passing through the lens portion RLn.

FIG. 7E is a diagram of the mask member M2 and the region of the lens portion RLn in FIG. 7D when viewed from the front. The mask member M2 has a pattern in which the outer half of the lens portion RLn is a transmitting region N2.

By using the mask member in this way, the number of divisions of the measurement region on the pupil of the eye to be examined can be practically increased without increasing the number of ring-shaped lens portions of the ring lens 125, and a more precise eye refractive power distribution can be obtained.

FIG. 8A and FIG. 8B are diagrams showing examples of the mask members of the mask 130 applied to the precision measurement mode of eye refractive power distribution. The mask member 137 in FIG. 8A, similar to FIG. 7C, has ring-shaped light shielding regions 137b and ring-shaped light transmitting regions 137a. The light shielding regions 137b are formed so that the outer half of the ring image for each of the lens portions RL1 to RL7 is not received by the imaging element 126. The light transmitting regions 137a are formed so that the inner half of the ring image for each of the lens portions RL1 to RL7 is received by the imaging element 126.

The mask member 138 in FIG. 8B, contrary to the mask member 137, has ring-shaped light transmitting regions 138a and ring-shaped light shielding regions 138b. The light transmitting regions 138a are formed, similar to FIG. 7E, so that the outer half of the ring image for each of the lens portions RL1 to RL7 is received by the imaging element 126. The light shielding regions 138b are formed so that the inner half of the ring image for each of the lens portions RL1 to RL7 is not received by the imaging element 126.

When executing the main measurement in the precision measurement mode of eye refractive power distribution, for example, the mask member 137 is inserted into the optical path first, allowing the inner half of the ring image of each of the ring-shaped lens portions RL1 to RL7 to be received by the imaging element 126, and the first image of the ring images is acquired by the control part 50. Next, the mask member 137 inserted into the optical path is switched to the mask member 138, and this time, the outer half of the ring image of each of the ring-shaped lens portions RL1 to RL7 is received by the imaging element 126, and the second image of the ring images is acquired by the control part 50. The control part 50 processes the acquired first image and determines the eye refractive power distribution in each meridian direction for the measurement region corresponding to the inner half of the ring width W of each of the lens portions RL1 to RL7. Additionally, the control part 50 processes the acquired second image and determines the eye refractive power distribution in each meridian direction for the measurement region corresponding to the outer half of the ring width W of each of the lens portions RL1 to RL7. Finally, the control part 50 combines the measurement results obtained by respective processing of the first image and the second image to obtain the eye refractive power distribution in the measurement region where the measurement region on the pupil corresponding to each of the lens portions RL1 to RL7 is divided into two parts. That is, the number of divisions in the measurement region divided by the lens portions RL1 to RL7 of the lens ring 125 can be effectively doubled, making it possible to obtain more precise measurement results of eye refractive power distribution. As a result, more accurate and more appropriate measurement results can be obtained.

While the above describes an example of dividing each measurement region corresponding to the lens portions RL1 to RL7 into two parts, the number of mask members of the mask 130 may be increased to further increase the number of divisions to three or more. This makes it possible to obtain eye refractive power distribution in even finer regions.

### <Measurement Region Limiting Measurement Mode>

The measurement region limiting measurement mode is a measurement mode that utilizes the mask members of the mask 130 to obtain eye refractive power limited to measurement regions corresponding to particular lens portions among the measurement regions on the pupil of the eye to be examined corresponding to the lens portions RL1 to RL7 of the ring lens 125. For example, in this measurement mode, the spherical power (S value), astigmatic power (C value), and astigmatic axis angle (A value) of the eye to be examined are obtained, and the measurement results thereof are used when prescribing eyeglasses or contact lenses.

For example, in the main measurement of the measurement region limiting measurement mode, the mask member 131 or 132 shown in FIG. 4A and FIG. 4B is used. In a case where the measurement region limiting measurement mode is selected, the control part 50 executes the main measurement with the mask member 131 or 132 of the mask 130 used in the preliminary measurement stage remaining in place. For example, in a case where the mask member 131 corresponds to the lens portion RL3, the diameter D4 of the center portion of the lens portion RL3 on the pupil is 3.2 mm, and the ring width W is 0.2 mm, the measurement region is limited to a pupil diameter of 3.0 mm to 3.4 mm.

The control part 50, at predetermined timing intervals, performs addition processing on the images of the ring images received on the imaging element 126 through the lens portion RL3, and acquires an image of the ring images with reduced noise light. The control part 50 thins the ring images of the acquired image, calculates the eye refractive power for each meridian direction, and performs predetermined processing on this eye refractive power to obtain the measurement results of the spherical power (S value), astigmatic power (C value), and astigmatic axis angle (A value) of the refractive error of the eye to be examined.

In a case where the eye to be examined has a small pupil with a pupil diameter of 3.0 mm or less, the mask member 132 is placed in the optical path, so that eye refractive power in the measurement region limited for small pupils can be obtained.

It might be possible that without using the mask member 131, the control part 50 can extract the ring image corresponding to the lens portion RL1 from among the seven ring images received on the imaging element 126 through the ring lens 125, and obtain the eye refractive power based on this ring image. However, as mentioned earlier, if the eye to be examined is a cataract eye with reflection and scattering in the eye, there is a possibility that the identification of the ring image corresponding to the lens portion RL1 may be affected. Regarding this, in a case of using this measurement region limiting measurement mode, while the measurement optical system has multiple ring lenses 125 arranged therein, the mask 130 can be appropriately switched to obtain the spherical power (S value), astigmatic power (C value), and astigmatic axis angle (A value) accurately and appropriately for prescribing eyeglasses or contact lenses.

### Variation Example of First Embodiment

Although the above describes that the precision measurement mode of eye refractive power distribution is separate from the cataract eye measurement mode of eye refractive power distribution, both modes may be combined. For example, in the application of the precision measurement mode, in a case where it is difficult to distinguish adjacent ring images due to the influence of reflection or scattering in the eye to be examined, the mask members 137 and 138 in FIG. 8A and FIG. 8B may be added to the mask members 135 and 136 in FIG. 6A and FIG. 6B, respectively. That is, for the ring images from the odd-numbered lens portions RL1, RL3, RL5, and RL7, a first image limited to the inner half of each ring image and a second image limited to the outer half of each ring image are acquired; and for the ring images from the even-numbered lens portions RL2, RL4, and RL6, a third image limited to the inner half of each ring image and a fourth image limited to the outer half of each ring image are acquired. Then, by combining the measurement results of the eye refractive power distribution of the first to fourth images, a more precise measurement result of eye refractive power distribution can be obtained even for a cataract eye with reflection or scattering in the eye.

Although the above describes the light receiving optical system 120 as a single optical system with the mask 130, the conversion member (ring lens 125), and the imaging element 126, the light receiving optical system 120 may also be configured with the mask 130, the conversion member (ring lens 125), and the imaging element 126 arranged respectively in two optical systems branched by a half mirror. In this case, by arranging different mask members in the mask 130 of each system, limitation states of ring images in different patterns can be created.

Further, although the above describes that the mask members of the mask 130 used are predetermined according to the measurement mode (in other words, predetermined ring images), the ring images received by the light receiving element (imaging element 126) may be arbitrarily selectable. For example, in a case where an electronic element such as a transmissive LCD is used as the mask 130, ring images can be arbitrarily selected by creating any mask state. For example, ring images can be arbitrarily selected by the examiner through operation of the operation part 17, which is an example of a selection part. For example, the examiner selects ring images (in other words, measurement, regions on the eye to be examined) by operating a screen displayed on the monitor 16. Then, the control part 50 controls the driving of the mask 130 so that the previously selected ring images are received by the imaging element 126 during measurement.

For example, in a case where the examiner wants to measure the center region, peripheral region, or specific measurement regions on the eye to be examined separately, since the measurement regions can be arbitrarily selected by arbitrarily selecting ring images, more appropriate eye refractive power information can be obtained according to the purpose. For example, in the measurement of an eye to be examined with a refractive multifocal IOL (IOL where eye refractive distribution changes in concentric circles) inserted, to measure the refractive power of a specific region, the ring images may be arbitrarily selected to obtain more appropriate eye refractive power information.

Additionally, for example, in a case where the eye to be examined has cataracts or the like, and noise appears in only some of the ring images, more appropriate eye refractive power information can be obtained by selecting ring images to exclude the noisy ring images. In this case, the control part 50 may automatically determine and select ring images to exclude noisy ring images based on the ring images obtained by the imaging element 126. Alternatively, the examiner may observe the image of the ring images, identify abnormal areas, exclude those rings, and then perform measurement again. If the noise portion can be identified, the mask is not necessarily circular.

Further, in a case where there is an alignment deviation (alignment deviation in XY direction) of the measurement part 12 (measurement optical system 100) with respect to the eye to be examined before and after insertion of the mask 130, or before and after switching of the mask 130, the position on the eye to be examined to which the mask 130 and ring images correspond changes. Therefore, the control part 50 may correct the measurement region where eye refractive power information is acquired based on that alignment deviation information. For example, in FIG. 6A and FIG. 6B, in a case where there is an alignment deviation during measurement when the mask is switched to obtain even-numbered ring images, compared to measurement masked to obtain odd-numbered ring images, the control part 50 takes into consideration the displacement ΔD of that alignment deviation and corrects the measurement region on the pupil corresponding to the ring images by the displacement ΔD when combining the eye refractive power information processed from each ring image sequentially received by the imaging element 126. Thereby, more appropriate measurement results (for example, output as a map of eye refractive power distribution) of the measurement region corresponding to the eye refractive power information can be obtained even in a case of an alignment deviation. The displacement ΔD is detected by the control part 50 based on the alignment index image in the anterior segment image captured by the imaging element 201.

Additionally, as the configuration of the mask member included in the mask 130 to prevent adjacent ring images from being received by the imaging element 126, the example described above uses two mask members 135 and 136 that limit to different ring images by skipping one (every other one) of multiple ring images, and there may be multiple mask members for sequentially limiting to different ring images by skipping two (every third one) or more of multiple ring images. However, the disclosure is not limited thereto.

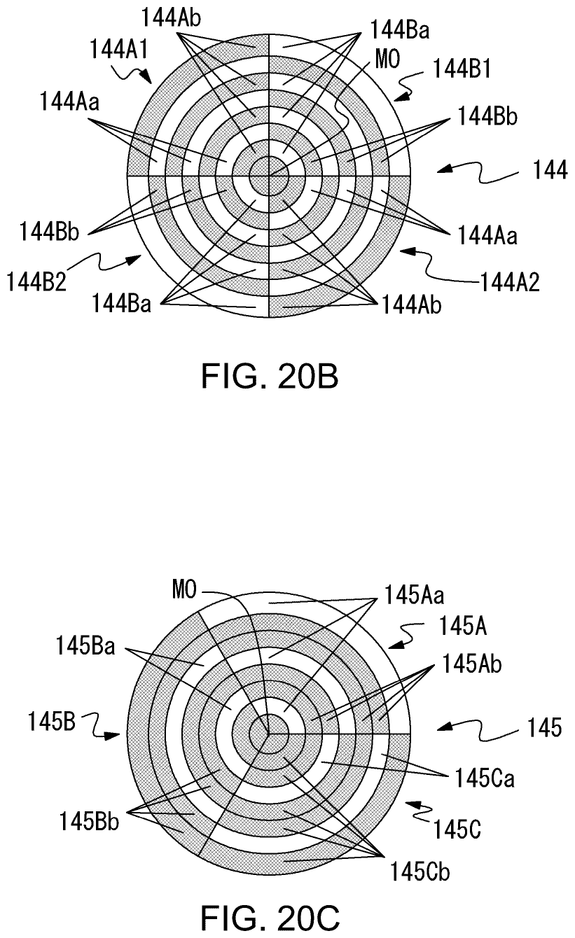

FIG. 20A, FIG. 20B, and FIG. 20C are diagrams explaining variation examples of the mask member that prevents adjacent ring images from being received by the imaging element 126. For example, the mask 143 of FIG. 20A is divided into two equal parts, a first region 143A and a second region 143B, in the circumferential direction of one circle corresponding to multiple ring images received by the imaging element 126, with reference to the center MO of the mask 143. In the upper half first region 143A, corresponding to multiple ring images received by the imaging element 126, semi-ring-shaped light transmitting regions 143Aa are formed to allow even-numbered ring images from the inside to be received by the imaging element 126, and semi-ring-shaped light shielding regions 143Ab are formed to prevent odd-numbered ring images from the inside from being received by the imaging element 126.

On the other hand, in the lower half second region 143B, corresponding to multiple ring images received by the imaging element 126, semi-ring-shaped light transmitting regions 143Ba are formed to allow odd-numbered ring images from the inside to be received by the imaging element 126, and semi-ring-shaped light shielding regions 143Bb are formed to prevent even-numbered ring images from the inside from being received by the imaging element 126.

Then, in the case of this variation example, the mask 143 is inserted into the optical path of the measurement optical system 100 (that is, the optical path of the light receiving optical system 120) by the mask drive part 130A, and is rotated by the angle of two equal divisions (in this example, 180 degrees which is ½ of 360 degrees) around the center MO, making it possible to change the limitation state of the ring images of the mask 143 in two patterns. The mask 143 is placed in the optical path so that the center MO thereof coincides with the optical axis L2.

During the measurement of eye refractive power, the control part 50 controls the driving of the mask drive part 130A, and for example, initially, the mask 143 is inserted into the optical path in the arrangement state shown in FIG. 20A and measurement is executed, whereby the ring images formed by the return light of the measurement light from the fundus, which passes through the light transmitting regions 143Aa and 143Ba of the mask 143, are received by the imaging element 126. The first image of the ring images at this time is stored in the memory 55. Next, the mask 143 is rotated by 180 degrees to change the limitation state of the ring images received by the imaging element 126. Then, the ring images under this limitation state are received by the imaging element 126, and the second image of the ring images is stored in the memory 55.

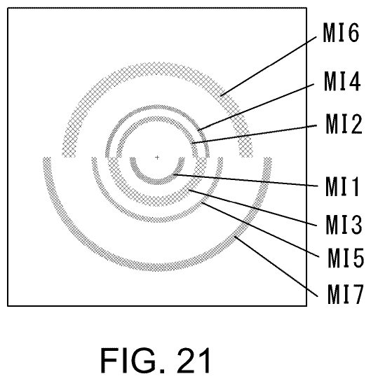

FIG. 21 is a diagram showing an example of the first image acquired with the mask 143 in the arrangement state of FIG. 20A, and is an example of the ring images acquired in a case of an eye to be examined with large aberration. In FIG. 21, in the upper half region, the second ring image MI2, the fourth ring image MI4, and the sixth ring image MI6 from the inside are received by the imaging element 126 due to the return light passing through the light transmitting region 143Aa. On the other hand, in the lower half region, the first ring image MI1, the third ring image MI3, the fifth ring image MI5, and the seventh ring image MI7 from the inside are received by the imaging element 126 due to the return light passing through the light transmitting region 143Ba.

Here, when focusing on the second ring image MI2 and the fourth ring image MI4 in the upper half region, and the third ring image MI3 in the lower half region, the width of the ring image MI3 is approximately the same as the width of the gap between the ring image MI2 and the ring image MI4. Therefore, if the mask 143 is not placed in the optical path and all the ring images are received by the imaging element 126, it will be difficult to distinguish the ring image MI3 from the adjacent ring image MI2 and ring image MI4. In contrast thereto, in a case where the mask 143 is used, adjacent ring images are not received by the imaging element 126, so it is easy to distinguish the ring image MI2, the ring image MI3, and the ring image MI4. That is, in FIG. 21, in the upper half region, there is a gap between the ring image MI2 and the ring image MI4 without the adjacent ring image MI3, making it possible to easily distinguish the ring image MI2 and the ring image MI4. Also, for the ring image MI3 in the lower half region, the adjacent ring image MI2 and ring image MI4 do not exist, and there are gaps between the ring image MI1 and the ring image MI5, making it possible to easily distinguish the ring image MI3.

Since the second image obtained by rotating the mask 143 by 180 degrees has the upper and lower regions of FIG. 21 reversed, the illustration thereof is omitted.

When the first image and the second image are obtained as described above, image processing respectively for the first image and the second image is performed by the control part 50, which also serves as the processor, so as to obtain measurement results of eye refractive power distribution based on the ring images of each image. Then, the overall measurement result of eye refractive power distribution is obtained by combining the measurement result based on the first image and the measurement result based on the second image.

Returning to the explanation of FIG. 20A to FIG. 20C, FIG. 20B is a diagram showing a variation example with respect to the mask member in FIG. 20A. The mask 144 shown in FIG. 20B is equally divided into four parts in the circumferential direction of one circle corresponding to multiple ring images received by the imaging element 126, and for each of the equally divided regions, ring-shaped light transmitting regions and light shielding regions are formed so that adjacent ring images are not received by the imaging element 126.

For example, in the mask 144 of FIG. 20B, with respect to the quadrant region 144A1 and the 180-degree opposite region 144A2, light transmitting regions 144Aa are formed so that even-numbered ring images from the inside are received by the imaging element 126, and light shielding regions 144Ab are formed to shield light so that odd-numbered ring images from the inside are not received by the light receiving element. In addition, with respect to the region 144B1 and the region 144B2 between the region 144A1 and the region 144A2, light transmitting regions 144Ba are formed so that odd-numbered ring images from the inside are received by the imaging element 126, and light shielding regions 144Bb are formed to shield light so that even-numbered ring images from the inside are not received by the light receiving element.

As this mask 144 is rotated by the mask drive part 130A by an angle corresponding to the number of equal divisions (in this example, 90 degrees) around the center MO of the mask 144, the limitation state of the ring images created by the mask member is changed in two patterns. Then, in response to this sequential change in the limitation state, the measurement results of eye refractive power distribution are obtained through respective image processing of the ring images sequentially received by the imaging element 126, and the overall eye refractive power is obtained by combining these measurement results.

In the examples shown above, the mask members of FIG. 20A and FIG. 20B are respectively divided into two equal parts and four equal parts in the circumferential direction of one circle corresponding to the ring images, but the number of divisions may be more than these as long as it is an even number. In other words, the mask member may be equally divided into an even number of two or more in the circumferential direction of one circle corresponding to multiple ring images received by the imaging element 126, and for each of these equally divided regions, the mask member includes first regions where even-numbered ring images from the inside are received by the imaging element 126 and odd-numbered ring images from the inside are shielded from being received by the imaging element 126, and second regions where odd-numbered ring images from the inside are received by the imaging element 126 and even-numbered ring images from the inside are shielded from being received by the imaging element 126, with the first regions and the second regions being arranged alternately.

FIG. 20C is a diagram showing another example of the mask member for preventing adjacent ring images from being received by the imaging element 126. The mask member may be equally divided into three or more parts in the circumferential direction of one circle corresponding to the ring images received by the imaging element 126, and according to the number of divisions, light transmitting regions and light shielding regions may be formed in each divided region so that adjacent ring images are not received by the imaging element 126.

For example, the mask 145 in FIG. 20C is an example in which the mask 145 is divided into three equal parts in the circumferential direction around the center MO. In the first region 145A which is the first one of the three equal divisions, light transmitting regions 145Aa are formed so that the first ring image from the inside and, corresponding to the number of divisions being three, the fourth and seventh ring images (skipping two ring images) are received by the imaging element 126, and light shielding regions 145Ab are formed to shield the second, third, fifth, and sixth ring images from the inside from being received. Further, in the second region 145B which is the second one of the three equal divisions, light transmitting regions 145Ba are formed so that, corresponding to the number of divisions being three, the second and fifth ring images from the inside are received by the imaging element 126, and light shielding regions 145Bb are formed to shield the first, third, fourth, sixth, and seventh ring images from the inside from being received. In addition, in the third region 145C which is the third one of the three equal divisions, light transmitting regions 145Ca are formed so that, corresponding to the number of divisions being three, the third and sixth ring images from the inside are received by the imaging element 126, and light shielding regions 145Cb are formed to shield the first, second, fourth, fifth, and seventh ring images from the inside from being received.

In the case of this example, the mask 145 is sequentially rotated by the mask drive part 130A by an angle corresponding to the number of divisions of three (in this example, 120 degrees), thereby changing the limitation state of the ring images created by the mask member in three patterns. Then, in response to this sequential change in the limitation state, the measurement results of eye refractive power distribution are obtained through respective image processing of the ring images sequentially received by the imaging element 126, and combined to obtain the overall eye refractive power. With such a mask member, in each image obtained by the imaging element 126, adjacent ring images are not received by the imaging element 126, so it is easy to distinguish ring images, and more accurate and more appropriate measurement results can be obtained.

In the case of the mask member shown in FIG. 20C, the interval between adjacent ring images in each acquired image can be set to skip two or more ring images, which is more advantageous when the number of ring images is increased and the interval between ring images is narrowed. In addition, as described above, the improved mask member that prevents adjacent ring images from being received by the imaging element 126 may be applied not only to a case where the eye to be examined is a cataract eye, but also to a case where the eye to be examined has large aberration.

The following summarizes the configurations of the mask members shown in FIG. 20A, FIG. 20B, and FIG. 20C. For example, the mask member may be a single mask material which is equally divided in the circumferential direction of one circle corresponding to multiple ring images received by the imaging element 126, and light shielding regions are formed corresponding to the equally divided regions to shield light so that at least adjacent ring images are not received by the imaging element 126. In this case, the mask member is rotatable around the center of the mask member corresponding to the ring images, and the limitation state of the ring images created by the mask member is sequentially changed by rotating the mask member based on an angle of equal divisions.

### Second Embodiment

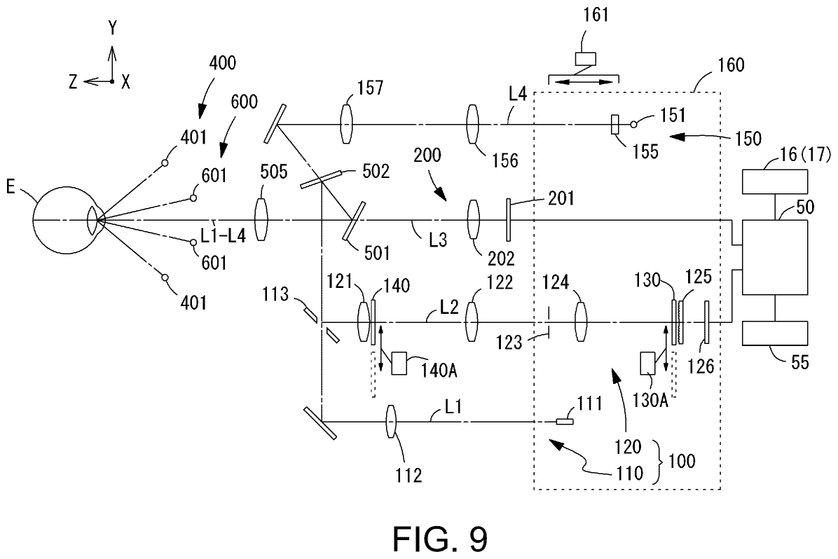

An example of the ophthalmic measurement device in the second embodiment will be described. FIG. 9 is a diagram showing the optical systems of the ophthalmic measurement device 10 in the second embodiment. The same elements as in the first embodiment are given the same reference numerals, and the description thereof will be omitted.

In a system utilizing multiple ring images, it is necessary to identify the number from the inside that each ring image received by the light receiving element corresponds to among the ring-shaped measurement regions on the eye to be examined. However, depending on the condition of the eye to be examined, the ring images on the light receiving element may be disturbed or the signal level may be low, making it difficult to distinguish the number that each ring image corresponds to. If the distinction between ring images is incorrect, it becomes difficult to obtain appropriate measurement results.

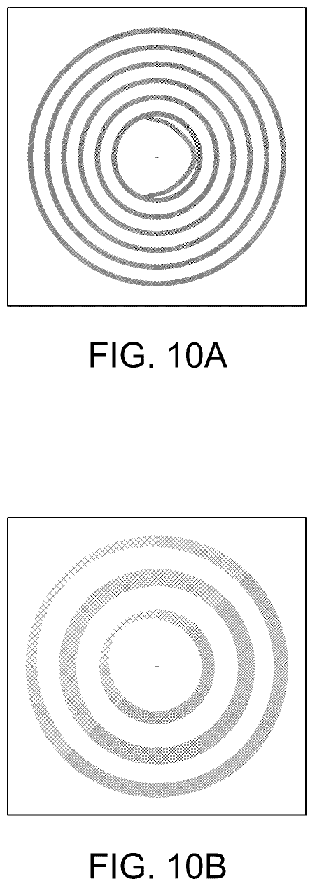

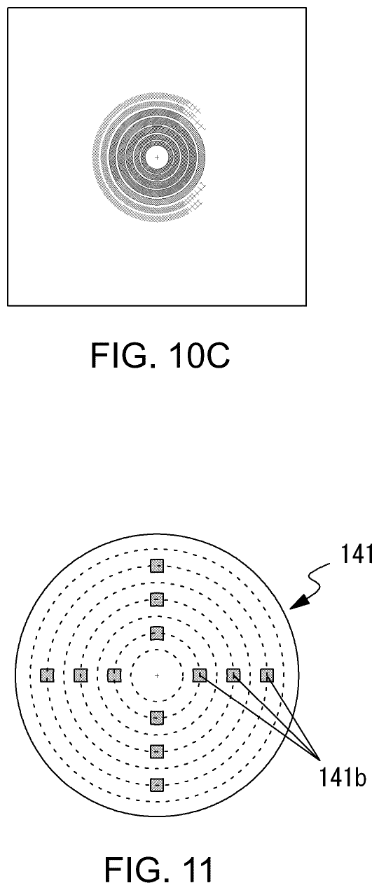

In a system utilizing multiple ring images, it is necessary to identify the number (in other words, circumferential position from the center position of the measurement optical axis) of each ring image received by the imaging element 126 among the measurement regions on the eye to be examined. However, the inventors' experiments have revealed that each ring image received by the imaging element 126 through the multiple ring lens 125 may, depending on the condition of the eye to be examined (for example, internal reflection in the eye, corneal reflection, etc.), be disturbed on the imaging element 126 or have a low signal level, making it difficult to distinguish the number of each ring image. For example, FIG. 10A shows an example of ring images in a case where internal reflection in the eye or corneal reflection overlaps. In this case, it is difficult to determine which of the lens portions RL1 and RL2 the two ring images from the inside correspond to, that is, from which position of the measurement regions on the eye to be examined, to which the adjacent ring images correspond, the light beam comes from.

Additionally, FIG. 10B and FIG. 10C show examples of ring images received by the imaging element 126 before diopter correction in which the driver 160 is moved. FIG. 10B shows a case where the spherical power of the eye to be examined is positive. In this case, the size of the ring images received by the imaging element 126 becomes larger, the number of ring images decreases, and the gradation of the ring images becomes lower. Therefore, it may become difficult to determine which of the lens portions RL1 to RL7 the ring images on the imaging element 126 correspond to, that is, from which position of the measurement region on the eye to be examined, to which each ring image corresponds, the light beam comes from. FIG. 10C shows a case where the spherical power of the eye to be examined is negative. In this case, the size of the ring images becomes smaller, and the ring images are received by the imaging element 126 with adjacent ring images overlapping each other. Therefore, it may become difficult to determine which of the lens portions RL1 to RL7 the ring images on the imaging element 126 correspond to, that is, from which position of the measurement region on the eye to be examined, to which each ring image corresponds, the light beam comes from.

Thus, the ophthalmic measurement device 10 in the second embodiment is an example that adds a mark formation mask 140, which is an example of the mark formation part (mark formation member), to the measurement optical system 100 so as to form marks in the measurement index image for distinguishing the lens portions RL1 to RL7 that the ring images received by the imaging element 126 correspond to. That is to say, the mark formation mask 140 forms marks in the measurement index image received by the imaging element 126 for distinguishing from which position of the measurement region on the eye to be examined, to which the measurement index image received by the imaging element 126 corresponds, the light beam comes from. For example, the mark formation mask 140 is arranged in the vicinity of or at the conjugate position of the ring lens 125. The vicinity of the ring lens 125 also includes cases where the light shielding portion of the mark formation mask 140 is directly formed on the ring lens 125. In the example of FIG. 9, the mark formation mask 140 is arranged at the conjugate position of the ring lens 125. The mark formation mask 140 is selectively inserted into and removed from the optical path by the mask drive part 140A. The driving of the mask drive part 140A is controlled by the control part 50.

The mark formation mask 140 may use, similar to the mask 130, a transparent material such as glass with a light shielding coating applied, or a sheet metal with physical holes may be used. In addition, an electronically controllable element such as a transmissive LCD may be used as the mask 140.

FIG. 11 is a diagram showing an example of a mask member 141 of the mark formation mask 140. For example, referring to FIG. 11, in the mask member 141, light shielding portions 141b for mark formation are provided on a light transmitting member 141a in portions corresponding to the even-numbered lens portions RL2, RL4, and RL6 from the inside of the ring-shaped lens portions RL1 to RL7 of the ring lens 125, so that different marks are formed between adjacent ring images. For example, the light shielding portions 141b are provided to form gaps in four directions at 0 degrees, 90 degrees, 180 degrees, and 270 degrees (directions at 90-degree intervals) so that cross-shaped gaps can be created for the ring images formed by the even-numbered lens portions RL2, RL4, and RL6 from the inside. Since it is sufficient that different marks are formed between adjacent ring images, in the example of the mask member 141, no light shielding portions are provided in portions corresponding to the odd-numbered lens portions RL1, RL3, RL5, and RL7 from the inside. Alternatively, the light shielding portions 141b may be provided in portions corresponding to the odd-numbered lens portions, and not for the even-numbered lens portions.

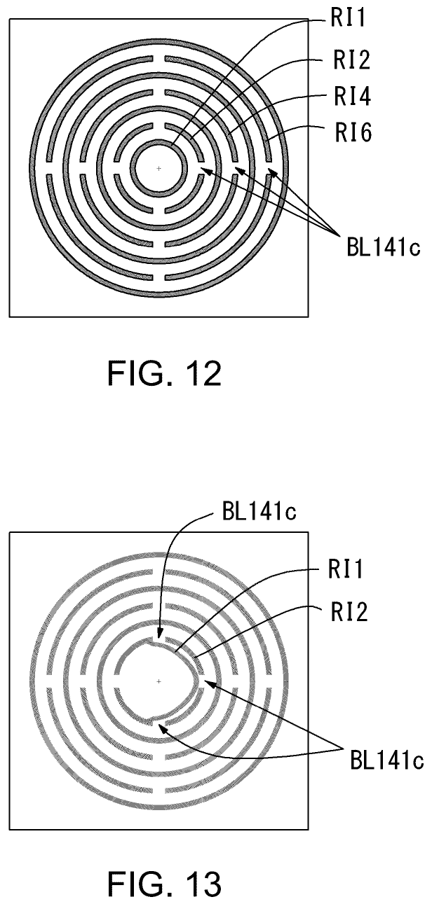

FIG. 12 shows an example of the ring images received by the imaging element 126 in a case where the mask member 141 is placed in the optical path of the light receiving optical system 120. In this case, marks BL141c of the gaps are formed at four locations in the up, down, right, and left directions in the second ring image RI2, fourth ring image RI4, and sixth ring image RI6 from the inside. As a result, for example, as shown in FIG. 13, even in a case where the first ring image RI1 from the inside approaches the second ring image RI2 and the signal level (gradation) of a part of the ring image RI1 is low, it is possible to distinguish which of the lens portions RL1 and RL2 each ring image corresponds to because the marks BL141c are formed in the second ring image RI2.

Without the marks BL141c, there is a possibility of confusing adjacent ring images (for example, the first and second ring images, the second and third ring images, and the third and fourth ring images). However, since the first and third ring images are sufficiently separated, the possibility of confusion is low. Therefore, even in a case where the marks BL141c are formed in every other ring image (skipping one) as shown in FIG. 12, it is possible to determine which of the lens portions RL1 to RL7 each ring image corresponds to, that is, which of the measurement regions on the eye to be examined each ring image corresponds to.

Although not shown, in a case where the spherical power of the eye to be examined is positive and the size of the ring images increases, resulting in a decrease in the number of ring images and lower gradation, and in a case where the spherical power of the eye to be examined is negative and the size of the ring images decreases, causing adjacent ring images to approach each other, it is possible to distinguish each ring image because the marks BL141c are formed in the second ring image RI2, the fourth ring image RI4, and the sixth ring image RI6, respectively.

The mark formation mask 140 as described above may be inserted into the light receiving optical system 120 for both the preliminary measurement and the main measurement in the first embodiment. Also, in a case where the mask 130 that limits the ring images to one as shown in FIG. 4A and FIG. 4B is used during the preliminary measurement, the mark formation mask 140 may be removed from the optical path, and the mark formation mask 140 may be inserted during the main measurement (for example, during the standard measurement mode of eye refractive power distribution).

For example, whether to insert the mark formation mask 140 into the optical path may be determined by the control part 50 based on the state of the ring images received by the imaging element 126, and the driving of the mask drive part 140A may be controlled based on the determination result. For example, in a case where the ring images received by the imaging element 126 are image-processed and it is determined that there is a portion that cannot be distinguished in each ring image, the mask drive part 140A is driven to insert the mark formation mask 140 into the optical path.

The control part 50 obtains the eye refractive power distribution based on the ring images received by the imaging element 126. Here, in a case where the mark formation mask 140 is inserted into the optical path, there is a missing portion in the ring image information due to the marks BL141c. In this case, the control part 50 obtains the information of the missing portion of the eye refractive power by interpolation based on the ring images surrounding the missing portion (for example, the surrounding ring images in the same ring images, excluding the missing portion).

### Variation Example of Second Embodiment

The example of the mask member 141 of the mark formation mask 140 is merely an example, and it is sufficient that different marks are formed between at least one pair of adjacent ring images so that at least one pair of adjacent ring images can be distinguished from each other. For example, light shielding portions may be provided for all ring images so as to form marks on adjacent ring images. For example, light shielding portions may be provided to form gaps in four directions at 45 degrees, 135 degrees, 215 degrees, and 305 degrees in the portions corresponding to the odd-numbered lens portions RL1, RL3, RL5, and RL7 from the inside, and to form gaps in four directions at 0 degrees, 90 degrees, 180 degrees, and 270 degrees in the portions corresponding to the even-numbered lens portions RL2, RL4, and RL6 from the inside. Alternatively, the number of gaps serving as marks may be different between adjacent ring images. In addition, the marks may have different gap shapes between adjacent ring images (for example, marks of triangular gaps facing the outside of the ring image and triangular gaps facing the inside of the ring image).

Furthermore, the mark formation part is not necessarily provided for all adjacent ring images to be distinguished. Since it is often relatively difficult to distinguish the ring images on the center portion side due to the condition of the eye to be examined, marks may be formed between at least some adjacent ring images, for example, so that the first and second ring images from the center side can be distinguished.

In addition, the mark formation part is not limited to a gap as long as the mark formation part serves as a mark for distinguishing from which position of the measurement region on the eye to be examined, to which the ring image corresponds, the light beam comes from. For example, while the mark formation part described above is arranged in the vicinity of or at the conjugate position of the ring lens 125, the mark formation part may be directly provided on the ring lens 125. In this case, the ring lens 125 itself may be modified to form marks on the ring lens 125. For example, a part of the ring-shaped columnar lens portion may have no radius of curvature. For example, a part of the ring-shaped columnar lens portion may be cut off, and a spherical lens portion may be formed there so that some of the ring images received by the imaging element 126 become point images.

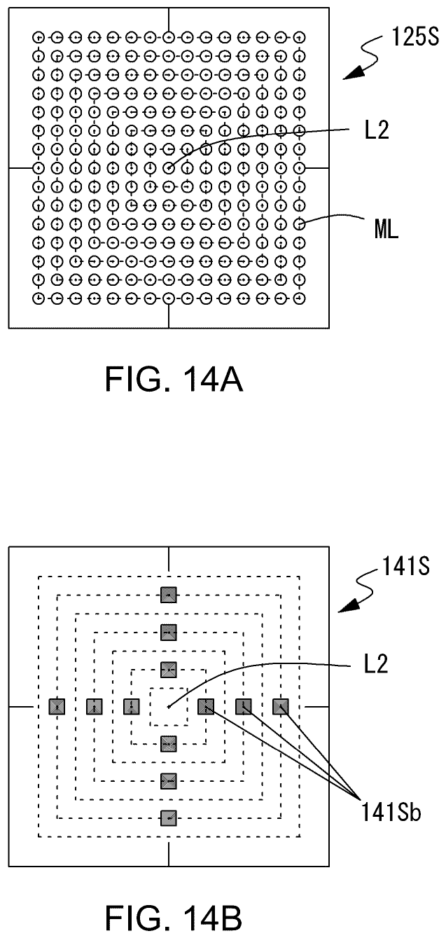

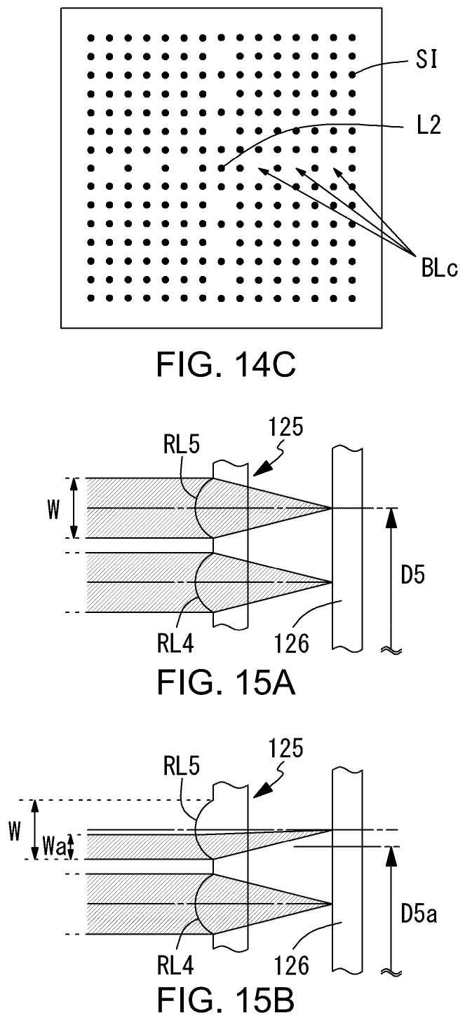

In addition, while the above describes a case in which the ring lens 125 is used as an example of the conversion member where the measurement index images received by the imaging element 126 become multiple ring images, a conversion member for converting into measurement index images composed of a large number of spot-like point images may be used. For example, FIG. 14A to FIG. 14C show an example where a microlens array for converting into measurement index images composed of a large number of point images is used instead of the ring lens 125, which is an example of a Hartmann plate used in a so-called Shack-Hartmann sensor.

FIG. 14A is a diagram showing an example of a microlens array 125S. A large number of microlenses ML are arranged from the first square to the seventh square in a grid pattern surrounding the center of the optical axis L2. FIG. 14B is an example of a mask member 141S arranged in the mark formation mask 140. The mask member 141S also has light shielding portions 141Sb provided on a light transmitting member. The light shielding portions 141Sb are provided to form gaps in the arrangement of point images in the second, fourth, and sixth squares of the arrangement of point images from the microlenses ML. For example, the light shielding portions 141Sb are arranged in four directions at 0 degrees, 90 degrees, 180 degrees, and 270 degrees centered on the optical axis L2, similar to the mask member 141 in FIG. 11.

FIG. 14C is a diagram showing point images SI received on the imaging element 162 through the microlens array 125S in a case where the mask member 141 is placed in the optical path. In this case, corresponding to the light shielding portions 141Sb, missing portions of point images SI occur in the second, fourth, and sixth squares of the arrangement of point images SI. Using these missing portions as marks makes it possible to distinguish to which square of the arrangement of microlenses ML each point image SI belongs.

### Third Embodiment

An example of the ophthalmic measurement device in the third embodiment will be described. Since the components of the ophthalmic measurement device 10 in the third embodiment are similar to the components in FIG. 1 and FIG. 2 of the first embodiment, the description thereof will be omitted. In the third embodiment, the mask 130 may not necessarily be arranged.

In a system utilizing ring images, the pupil edge (iris edge) may overlap with the width of the corresponding ring-shaped measurement region on the pupil of the eye to be examined, causing vignetting of the measurement light beam passing through the pupil (measurement region). In this case, if the vignetting of the measurement light beam is not taken into consideration, accurate eye refractive power information at the measurement position (measurement region) on the pupil cannot be obtained. Similar problems exist in a system that utilizes a large number of point images around the measurement optical axis.

FIG. 15A and FIG. 15B are diagrams explaining measurement considering the vignetting state of the measurement light beam due to the pupil edge. FIG. 15A shows the passage of the measurement light beam (reflected light from the fundus) and imaging on the imaging element 126 in a case where there is no vignetting of the measurement light beam due to the pupil edge, for the ring-shaped lens portions RL4 and RL5 (the lens portions are merely examples, and are not limited to these) of the ring lens 125. In this case, the eye refractive power of the measurement region on the pupil corresponding to the regions of the lens portions RL4 and RL5 can be obtained.

On the other hand, FIG. 15B shows the passage of the measurement light beam and imaging on the imaging element 126 in a case where vignetting of the measurement light beam due to the pupil edge occurs in the measurement region on the pupil corresponding to the region of the lens portion RL5. In both cases of FIG. 15A and FIG. 15B, the imaging position on the imaging element 126 of the measurement light beam that could pass through the lens portion RL5 is the same. Therefore, it is not possible to determine whether vignetting of the measurement light beam is occurring due to the pupil edge simply by analyzing the ring image formed (received) on the imaging element 126.

Thus, the anterior segment image acquired by the observation optical system 200 is used. For example, the vignetting state of the measurement light beam in the measurement region is detected (determined) by comparing the pupil region in the anterior segment image with the measurement region on the pupil corresponding to each lens portion of the ring lens 125. Then, the measurement region (or measurement position) where the eye refractive power is obtained is corrected based on the result.

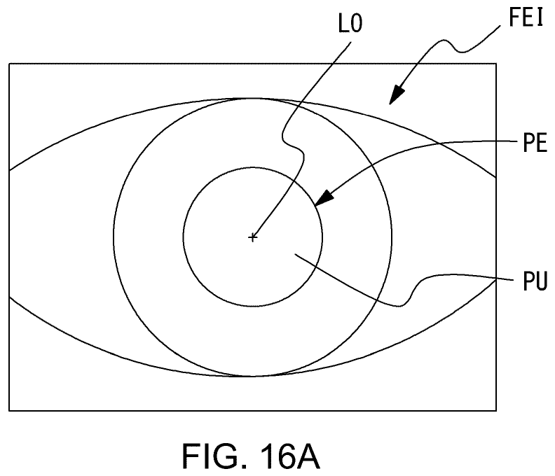

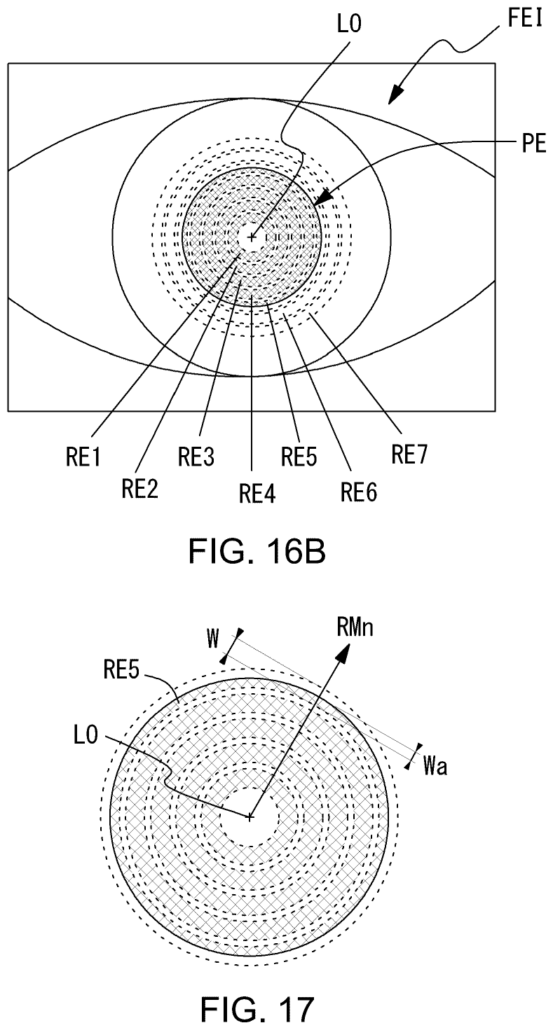

FIG. 16A is a diagram showing an example of an anterior segment image FEI acquired by the observation optical system 200 during execution of the main measurement of eye refractive power distribution. FIG. 16B is a diagram showing the positional relationship between the pupil PU in the anterior segment image FEI of FIG. 16A and the measurement region of each lens portion (RL1 to RL7) of the ring lens 125 corresponding on the pupil.

The control part 50 performs image processing on the anterior segment image FEI obtained by the imaging element 201 of the observation optical system 200, and obtains, for example, the position (for example, coordinate position) of the pupil edge PE of the pupil PU with reference to the center position L0 where the optical axis L3 of the measurement optical system 100 is located. Next, as shown in FIG. 16B, the control part 50 corresponds the measurement regions RE1, RE2, RE3, RE4, RE5, RE6, and RE7 of the lens portions (RL1 to RL7) of the ring lens 125 on the anterior segment image FEI with reference to the center position L0. These multiple measurement regions RE1 to RE7 can be rephrased as regions divided in the meridian direction with reference to the measurement optical axis.

Next, the control part 50 compares the pupil edge PE with each measurement region (RE1 to RE7) and detects the vignetting state of the measurement light beam in the meridian direction for each measurement region (RE1 to RE7). In the example of FIG. 16B, since the pupil edge PE overlaps with the interior (a part) of the measurement region RE5, it is detected that vignetting exists in a part of the measurement light beam passing through the measurement region RE5 of the lens portion RL5.

The control part 50 performs image processing on each ring image received by the imaging element 126, and as shown in FIG. 17, obtains eye refractive power information for each meridian direction RMn for the measurement region corresponding to each ring image. In the example of FIG. 17, the ring images corresponding to the measurement regions RE6 and RE7 are not received by the imaging element 126, and the ring images corresponding to the measurement regions RE1 to RE5 are received by the imaging element 126. At this time, the imaging position on the imaging element 126 of the ring image formed by the measurement light beam passing through the measurement region RE5 is the same as in the case where there is no vignetting in the measurement light beam. Therefore, the control part 50 obtains eye refractive power information for each meridian direction RMn by image-processing the ring images regardless of the presence or absence of vignetting of the measurement light beam. However, regarding the measurement region of that measurement result, the control part 50 interprets and outputs it as a measurement region with the remaining region width Wa obtained by subtracting the vignetting portion of the measurement light beam due to the pupil edge PE from the ring width W, rather than a region with the ring width W (the ring width on the pupil corresponding to the lens portion R5).

For example, in FIG. 15A described above, in a case where the center diameter D5 of the ring-shaped lens portion RL5 corresponding to the measurement region RE5 is 5 mm, the ring width W is 0.2 mm, and there is no vignetting of the measurement light beam, the measurement result of eye refractive power distribution for each meridian direction RMn based on the ring image from the lens portion RL5 is output as a measurement result for a measurement position with a pupil diameter of 5 mm. The measurement result is also expressed as a measurement result for a measurement region with a pupil diameter of 4.8 mm to 5.2 mm.

On the other hand, as shown in FIG. 15B described above, if there is vignetting of the measurement light beam passing through the measurement region RE5, and the region width Wa through which the measurement light beam passes is 0.1 mm, the measurement result based on that ring image is corrected to a measurement result for a measurement position with a pupil diameter D5a of 4.9 mm, based on the relationship between diameter D5, ring width W, and region width Wa. Also, the measurement result is corrected to a measurement result for a measurement region with a pupil diameter of 4.8 mm to 5.0 mm. Thereby, it is possible to obtain a more appropriate measurement result at a more accurate measurement position even in a case where there is vignetting of the measurement light beam due to the pupil edge.

In a case where the pupil edge PE is decentered with respect to the measurement optical axis L3, the measurement result based on the ring image may be corrected by calculating the remaining region width Wa obtained by subtracting the vignetting portion of the measurement light beam for each meridian direction RMn, and correcting to a measurement result for a measurement position or measurement region according to that region width Wa. Thereby, it is possible to obtain a more accurate and more appropriate measurement result even in a case where the pupil edge PE is decentered.

For example, the measurement result of the eye refractive power distribution is displayed as a color map, a contour map, or the like that is color-coded according to the magnitude of refractive power. For example, the color map expresses the eye refractive power distribution for a two-dimensional position on the anterior segment of the eye to be examined, as viewed from the front, by color-coding. In this case, as the measurement position or measurement region of the eye refractive power distribution is corrected more accurately, a more appropriate measurement result can be provided.

While the third embodiment described above explains a case of using the ring lens 125, the third embodiment can be similarly applied to cases where a microlens array or a Hartmann plate of a Shack-Hartmann sensor is used instead of the ring lens 125 to receive a large number of point images around the measurement optical axis on the imaging element 126.

### Variation Example of Third Embodiment

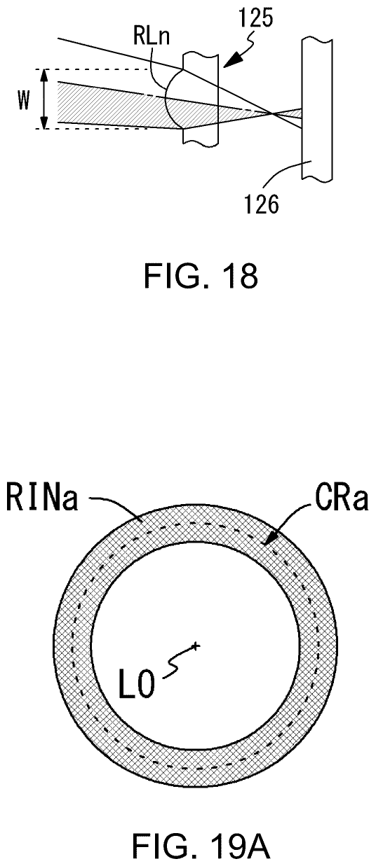

In the explanation of FIG. 15A and FIG. 15B above, it is assumed that the ring image formed by the measurement light beam that could pass through the pupil is imaged at the same position on the imaging element 126 in both a case where there is vignetting of the measurement light beam and a case where there is no vignetting. However, in reality, the imaging element 126 is not necessarily positioned at the focusing point of the ring image, and as shown in FIG. 18, the ring image on the imaging element 126 may be blurred. FIG. 18 is a diagram explaining a case where vignetting of the measurement light beam occurs due to the pupil edge in the measurement region on the pupil corresponding to a certain ring-shaped lens portion RLn of the ring lens 125, which is a case where the imaging element 126 is located at a position away from and behind the focusing point of the ring image. For example, it is assumed that the outer half of the measurement light beam passing through the measurement region on the pupil has vignetting due to the pupil edge. This vignetting state of the measurement light beam is detected by comparing the pupil region in the anterior segment image obtained by the observation optical system 200 with the measurement region on the pupil corresponding to each lens portion of the ring lens 125.

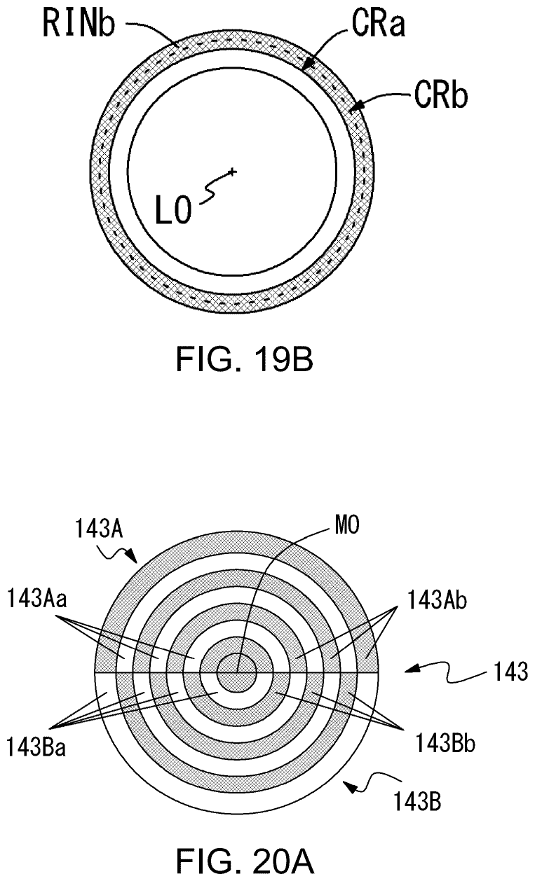

FIG. 19A is a diagram showing a ring image detected on the image acquired by the imaging element 126 in a case where no vignetting occurs in the measurement light beam passing through the pupil in FIG. 18. On the other hand, FIG. 19B is a diagram showing a ring image detected on the image acquired by the imaging element 126 in a case where vignetting occurs in the measurement light beam passing through the pupil in FIG. 18.

For example, as shown in FIG. 19A, in a case where no vignetting occurs in the measurement light beam, the eye refractive power is obtained (calculated) based on the center position CRa of the width of the ring image RINa detected by processing the image acquired by the imaging element 126. In contrast, as shown in FIG. 19B, in a case where vignetting occurs in the measurement light beam, the eye refractive power of the measurement region on the pupil corresponding to the ring image is obtained based on the detection result of the vignetting state of the measurement light beam and the detection position of the ring image RINb, rather than the center position CRb of the width of the ring image RINb detected by processing the image acquired by the imaging element 126. For example, the eye refractive power equivalent to the center position CRa of the ring width in a case where no vignetting occurs in the measurement light beam is obtained based on the relationship between the detection result of the vignetting state of the measurement light beam and the detection position of the ring image RINb. For example, as shown in FIG. 18, in a case where the outer half of the measurement light beam has vignetting due to the pupil edge, the center position CRa is determined by doubling the ring width of the ring image RINb inward, and the eye refractive power for this center position CRa is obtained. Alternatively, a function or table defining the correspondence relationship between the vignetting state of the measurement light beam and the eye refractive power for the position of the ring image may be stored in a storage part (for example, memory 55), and the eye refractive power may be obtained based on this. The eye refractive power distribution is determined for each meridian direction with reference to the center position L0 through which the measurement optical axis passes.

With such processing, even in a case where the focusing point of the ring image is not positioned on the imaging element 126 and the ring image is blurred, more appropriate eye refractive power can be obtained with the information of the vignetting state of the measurement light beam.

1. An ophthalmic measurement device for measuring eye refractive power distribution of an eye to be examined, the ophthalmic measurement device comprising:
a measurement optical system, including a light projecting optical system that projects a measurement light onto a fundus of the eye to be examined, and a light receiving optical system that receives a reflected light of the measurement light from the fundus of the eye to be examined by a light receiving element;
a conversion member, arranged at a pupil conjugate position of the eye to be examined in the measurement optical system, and converting the reflected light from the fundus into a plurality of ring images and causing the ring images to be received by the light receiving element;
a limiting part, including a limiting member arranged at a conjugate position of the conversion member or in a vicinity of the conversion member, and changing a measurement region on the eye to be examined by limiting, with the limiting member, some of the ring images received by the light receiving element;
a limiting controller, configured to control the limiting part and cause the measurement region on the eye to be examined that is changed by the limiting member to change in at least two patterns; and
a processor, configured to process the ring images received by the light receiving element and obtain eye refractive power information.

2. The ophthalmic measurement device according to claim 1, wherein
the limiting part is configured so that the limiting member is changeable to a limitation state of at least two patterns so that, among the plurality of ring images received by the light receiving element, adjacent ring images are not received by the light receiving element,
the limiting controller is configured to sequentially change the limitation state of the limiting member, and
the processor is configured to obtain eye refractive power information of the plurality of ring images by combining measurement results of eye refractive power obtained by respectively processing the ring images sequentially received by the light receiving element through sequential change of the limitation state.

3. The ophthalmic measurement device according to claim 1, wherein
the limiting part is configured so that the limiting member is changeable to a limitation state in which a ring width of the measurement region on the eye to be examined corresponding to each ring image received on the light receiving element is divided into at least two parts,
the limiting controller is configured to sequentially change the limitation state divided into at least two parts, and
the processor is configured to obtain eye refractive power information of the plurality of ring images by combining measurement results of eye refractive power obtained by respectively processing the ring images sequentially received by the light receiving element through sequential change of the limitation state.

4. The ophthalmic measurement device according to claim 1, wherein
the limiting part is configured to be capable of arbitrarily selecting the ring images received by the light receiving element from among the plurality of ring images that are receivable on the light receiving element, and
the limiting controller is configured to control the limiting part so that the ring images preselected are received by the light receiving element.

5. The ophthalmic measurement device according to claim 2, wherein
the limiting member comprises a plurality of limiting members that sequentially limit the ring images received by the light receiving element to different ring images, by skipping one or skipping two or more, corresponding to the plurality of ring images that are receivable by the light receiving element.

6. The ophthalmic measurement device according to claim 2, wherein
the limiting member is equally divided in a circumferential direction of one circle corresponding to the plurality of ring images received by the light receiving element, and corresponding to equally divided regions, light shielding regions are formed to shield light so that at least adjacent ring images are not received by the light receiving element,
the limiting part comprises a rotation part that rotates the limiting member around a center of the limiting member corresponding to the ring images, and
the limiting controller is configured to control the rotation part and rotate the limiting member based on an angle of equal division to sequentially change the limitation state of the ring images created by the limiting member.

7. The ophthalmic measurement device according to claim 6, wherein
the limiting member is equally divided into an even number of two or more in a circumferential direction of one circle corresponding to the plurality of ring images received by the light receiving element, and for equally divided regions, a first region and a second region are alternately arranged,
wherein
the first region causes even-numbered ring images from inside to be received by the light receiving element and shields light so that odd-numbered ring images from inside are not received by the light receiving element, and
the second region causes odd-numbered ring images from inside to be received by the light receiving element and shields light so that even-numbered ring images from inside are not received by the light receiving element, and
the limiting controller is configured to control the rotation part and rotates the limiting member by an angle of equal division to sequentially change the limitation state of the ring images created by the limiting member.

8. The ophthalmic measurement device according to claim 6, wherein
the limiting member is equally divided into three or more parts in a circumferential direction of one circle corresponding to the plurality of ring images received by the light receiving element, and according to the number of divisions, light transmitting regions and light shielding regions are formed in each divided region so that adjacent ring images are not received by the light receiving element, and
the limiting controller is configured to control the rotation part and sequentially rotate the limiting member by an angle of equal division to sequentially change the limitation state of the ring images created by the limiting member.

9. The ophthalmic measurement device according to claim 1, further comprising:
a diopter corrector, configured to adjust an imaging state of the ring images received by the light receiving element according to a diopter of the eye to be examined;
a measurement controller, configured to perform premeasurement that obtains the diopter of the eye to be examined to operate the diopter corrector, and perform main measurement by operating the diopter corrector based on a measurement result obtained by premeasurement; and
a mode switching part, configured to switch between a first mode in which measurement is performed without using the limiting part during main measurement, and a second mode in which measurement is performed using the limiting part during at least one of premeasurement and main measurement.

10. The ophthalmic measurement device according to claim 1, wherein
the limiting controller is configured to:
control the limiting part to reduce the ring images that are receivable on the light receiving element, among the plurality of ring images received on the light receiving element, during premeasurement that obtains a diopter of the eye to be examined, and
control the limiting part so that the measurement region on the eye to be examined changes, compared to during premeasurement, during main measurement after premeasurement.

11. The ophthalmic measurement device according to claim 1, further comprising:
a mark formation part, configured to form a mark in a measurement index image to distinguish from which position of the measurement region on the eye to be examined, to which the measurement index image received by the light receiving element corresponds, a light beam comes from,
wherein the mark formation part is arranged in the vicinity of or at the conjugate position of the conversion member, or is provided on the conversion member.

12. The ophthalmic measurement device according to claim 11, wherein
the mark formation part is configured to form different marks between at least one pair of adjacent ring images of the measurement index image.

13. The ophthalmic measurement device according to claim 1, further comprising:
an anterior segment image acquisition part, configured to acquire an anterior segment image including a pupil of the eye to be examined,
wherein the processor is configured to detect a vignetting state due to a pupil edge of a measurement light beam passing through the measurement region by comparing a pupil region in the anterior segment image with the measurement region corresponding on the pupil, and correct the measurement region for obtaining eye refractive power information based on a detection result.

14. The ophthalmic measurement device according to claim 13, wherein
the processor is configured to obtain eye refractive power information in the measurement region based on a reception result of a measurement index image received by the light receiving element regardless of presence or absence of vignetting of the measurement light beam passing through the measurement region, and with respect to eye refractive power information of a first measurement region where vignetting of the measurement light beam is detected, replace the first measurement region with a second measurement region that corrects the first measurement region based on a vignetting state of the measurement light beam passing through the first measurement region.

15. The ophthalmic measurement device according to claim 14, wherein
the processor is configured to obtain eye refractive power information for each meridian direction with reference to a measurement optical axis of the measurement optical system, and determine the second measurement region by a remaining region width obtained by subtracting a vignetting portion of the measurement light beam from the first measurement region for each meridian direction.
